SEQUENCE LISTING (12 sequences)

SEQ ID NO: 1 - DNA, 2389 bp, Homo sapiens
gggaaggcgagcagtgccaatctacagcgaagaaagtctcgtttggtaaaagcgagaggggaaagcctgagcatgcagagtgtgcagagcacgagcttttgtctccgaaagcagtgcctttgcctgaccttcctgcttctccatctcctgggacaggtcgctgcgactcagcgctgccctccccagtgcccgggccggtgccctgcgacgccgccgacctgcgcccccggggtgcgcgcggtgctggacggctgctcatgctgtctggtgtgtgcccgccagcgtggcgagagctgctcagatctggagccatgcgacgagagcagtggcctctactgtgatcgcagcgcggaccccagcaaccagactggcatctgcacggcggtagagggagataactgtgtgttcgatggggtcatctaccgcagtggagagaaatttcagccaagctgcaaattccagtgcacctgcagagatgggcagattggctgtgtgccccgctgtcagctggatgtgctactgcctgagcctaactgcccagctccaagaaaagttgaggtgcctggagagtgctgtgaaaagtggatctgtggcccagatgaggaggattcactgggaggccttacccttgcagcttacaggccagaagccaccctaggagtagaagtctctgactcaagtgtcaactgcattgaacagaccacagagtggacagcatgctccaagagctgtggtatggggttctccacccgggtcaccaataggaaccgtcaatgtgagatgctgaaacagactcggctctgcatggtgcggccctgtgaacaagagccagagcagccaacagataagaaaggaaaaaagtgtctccgcaccaagaagtcactcaaagccatccacctgcagttcaagaactgcaccagcctgcacacctacaagcccaggttctgtggggtctgcagtgatggccgctgctgcactccccacaataccaaaaccatccaggcagagtttcagtgctccccagggcaaatagtcaagaagccagtgatggtcattgggacctgcacctgtcacaccaactgtcctaagaacaatgaggccttcctccaggagctggagctgaagactaccagagggaaaatgtaacctatcactcaagaagcacacctacagagcacctgtagctgctgcgccacccaccatcaaaggaatataagaaaagtaatgaagaatcacgatttcatccttgaatcctatgtattttcctaatgtgatcatatgaggacctttcatatctgtcttttatttaacaaaaaatgtaattaactgtaaacttggaatcaaggtaagctcaggatatggcttaggaatgacttactttcctgtggttttattacaaatgcaaatttctataaatttaagaaaacaagtatataatttactttgtagactgtttcacattgcactcatcatattttgttgtgcactagtgcaattccaagaaaatatcactgtaatgagtcagtgaagtctagaatcatacttaacatttcattgtacaagtattacaaccatatattgaggttcattgggaagattctctattggctccctttttgggtaaaccagctctgaacttccaagctccaaatccaaggaaacatgcagctcttcaacatgacatccagagatgactattacttttctgtttagttttacactaggaaacgtgttgtatctacagtaatgaaatgtttactaagtggactggtgtcataaactttctccatttaagacacattgactcctttccaatagaaagaaactaaacagaaaactcccaatacaaagatgactggtccctcatagccctcagacatttatatattggaagctgctgaggcccccaagttttttaattaagcagaaacagcatattagcagggattctctcatctaactgatgagtaaactgaggcccaaagcacttgcttacatcctctgatagctgtttcaaatgtgcattttgtggaattttgagaaaaatagagcaaaatcaacatgactggtggtgagagaccacacattttatgagagtttggaattattgtagacatgcccaaaacttatccttgggccataattatgaaaactcatgatcaagatatatgtgtatacatacatgtatctggtttgtcaggctacaaggtaggctgcaaaattaaatctagacattcttttaatgccaccacacgtgttccgcttctctcttttaaagtatttataaaaatataaattgtacattttgtaaaatattatgtttgatttctctacttgtcatatcactaaataaacacgattttattgctgaaaaaaaaaaaaaaaaaaa

SEQ ID NO: 2 - DNA, 768 bp, Homo sapiens
acggcggtagagggagataactgtgtgttcgatggggtcatctaccgcagtggagagaaatttcagccaagctgcaaattccagtgcacctgcagagatgggcagattggctgtgtgccccgctgtcagctggatgtgctactgcctgagcctaactgcccagctccaagaaaagttgaggtgcctggagagtgctgtgaaaagtggatctgtggcccagatgaggaggattcactgggaggccttacccttgcagcttacaggccagaagccaccctaggagtagaagtctctgactcaagtgtcaactgcattgaacagaccacagagtggacagcatgctccaagagctgtggtatggggttctccacccgggtcaccaataggaaccgtcaatgtgagatgctgaaacagactcggctctgcatggtgcggccctgtgaacaagagccagagcagccaacagataagaaaggaaaaaagtgtctccgcaccaagaagtcactcaaagccatccacctgcagttcaagaactgcaccagcctgcacacctacaagcccaggttctgtggggtctgcagtgatggccgctgctgcactccccacaataccaaaaccatccaggcagagtttcagtgctccccagggcaaatagtcaagaagccagtgatggtcattgggacctgcacctgtcacaccaactgtcctaagaacaatgaggccttcctccaggagctggagctgaagactaccagagggaaaatgtaa

SEQ ID NO: 3 - DNA, 948 bp, Homo sapiens
tgccctgcgacgccgccgacctgcgcccccggggtgcgcgcggtgctggacggctgctcatgctgtctggtgtgtgcccgccagcgtggcgagagctgctcagatctggagccatgcgacgagagcagtggcctctactgtgatcgcagcgcggaccccagcaaccagactggcatctgcacggcggtagagggagataactgtgtgttcgatggggtcatctaccgcagtggagagaaatttcagccaagctgcaaattccagtgcacctgcagagatgggcagattggctgtgtgccccgctgtcagctggatgtgctactgcctgagcctaactgcccagctccaagaaaagttgaggtgcctggagagtgctgtgaaaagtggatctgtggcccagatgaggaggattcactgggaggccttacccttgcagcttacaggccagaagccaccctaggagtagaagtctctgactcaagtgtcaactgcattgaacagaccacagagtggacagcatgctccaagagctgtggtatggggttctccacccgggtcaccaataggaaccgtcaatgtgagatgctgaaacagactcggctctgcatggtgcggccctgtgaacaagagccagagcagccaacagataagaaaggaaaaaagtgtctccgcaccaagaagtcactcaaagccatccacctgcagttcaagaactgcaccagcctgcacacctacaagcccaggttctgtggggtctgcagtgatggccgctgctgcactccccacaataccaaaaccatccaggcagagtttcagtgctccccagggcaaatagtcaagaagccagtgatggtcattgggacctgcacctgtcacaccaactgtcctaagaacaatgaggccttcctccaggagctggagctgaagactaccagagggaaaatgtaa

SEQ ID NO: 4 - protein, 357 aa, Homo sapiens
MQSVQSTSFCLRKQCLCLTFLLLHLLGQVAATQRCPPQCPGRCPATPPTCAPGVRAVLDGCSCCLVCARQRGESCSDLEPCDESSGLYCDRSADPSNQTGICTAVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM

SEQ ID NO: 5 - protein, 254 aa, Homo sapiens
AVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM

SEQ ID NO: 6 - protein, 315 aa, Homo sapiens
CPATPPTCAPGVRAVLDGCSCCLVCARQRGESCSDLEPCDESSGLYCDRSADPSNQTGICTAVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM

SEQ ID NO: 7 - DNA, 23 bp, Artificial Sequence
  Primer sequence 1
aagtcaagtctctgcatctctgc

SEQ ID NO: 8 - DNA, 23 bp, Artificial Sequence
  Primer sequence 2
ctgagcacctgttaaatttctcc

SEQ ID NO: 9 - DNA, 21 bp, Artificial Sequence
  SiRNA sequence 1
gcagaguucaaaagcccuutt

SEQ ID NO: 10 - DNA, 21 bp, Artificial Sequence
  SiRNA sequence 2
aagggcuuuugaacucugctt

SEQ ID NO: 11 - DNA, 21 bp, Artificial Sequence
  siRNA sequence 3
uuguacggcaucagcguuatt

SEQ ID NO: 12 - DNA, 21 bp, Artificial Sequence
  siRNA sequence 4
uuacgcugaugccguacaatt

(19) United States
(12) Patent Application Publication
(10) Pub. No.: US 2010/0004169 A1
(43) Pub. Date: Jan. 7, 2010

(54) CCN3 peptide

(75) Inventors: Alexandra Elizabeth Irvine, Belfast (GB); Lynn McCallum, Belfast (GB); Bernard Perbal, Cranford, NJ (US); Anthony Whetton, Manchester (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Queen's University of Belfast, Belfast (GB)

(21) Appl. No.: 12/311,197

(22) PCT Filed: Apr. 30, 2007
(86) PCT No.: PCT/GB2007/001592

§ 371 Date: Jul. 15, 2009

(30) Foreign Application Priority Data
Sep. 22, 2006 (GB) 0618748.8

(51) Int. Cl. A61K 38/16; G01N 33/53; C07H 21/00; C07K 14/00; C12N 15/74; C12N 5/10; A61P 35/00

(52) U.S. Cl. 514/12; 435/7.1; 536/23.1; 530/399; 435/320.1; 435/358

(57) ABSTRACT

The present application relates to nucleic acid and peptide sequences of CCN3 and derivatives and fragments thereof useful in the treatment of disease, in particular tumours and/or for use as a clinical marker.

### FIELD OF INVENTION

The present invention relates to nucleic acid and peptide sequences useful in the treatment of tumours and/or as clinical markers. In particular, the present invention relates to partial nucleic acid sequences and peptide sequences of CCN3 useful in reducing tumour colony formation and/or cell proliferation in Chronic myeloid leukaemia.

### BACKGROUND TO THE INVENTION

Chronic myeloid leukaemia (CML) is a clonal disorder of pluripotent hematopoietic cells that is characterized by the presence of the Philadelphia chromosome (PH⁺), the result of a reciprocal translocation between chromosome 9 and 22. The translocation encodes a chimeric protein, BCR-ABL, which is a constitutively activated protein tyrosine kinase (PTK) and which has an essential role in the molecular pathology of CML.

To date, an Abl tyrosine kinase inhibitor has been used to treat CML. However, there is clear evidence of resistance to this drug.

There is a need to develop other approaches to treat the Chronic myeloid leukaemia (CML).

### SUMMARY OF INVENTION

The inventors have determined that a growth regulator, CCN3 is down regulated and phosphorylated as a result of BCR-ABL kinase activity. Furthermore, the inventors have determined that a relationship exists between the level of CCN3 and BCR-ABL kinase activity.

Accordingly, a first aspect of the present invention provides a method of modulating BCR-ABL activity in a cell said method comprising providing an agent to said cell to modulate the level or activity of CCN3 or a derivative thereof, thereby modulating the level or activity of BCR-ABL.

CCN3 includes CCN3 protein/polypeptide or a fragment thereof, or CCN3 nucleic acid which encodes CCN3 polypeptide or a fragment thereof.

The level or activity of CCN3 can be modulated at any level including transcription, protein expression, localisation of CCN3 protein in the cell, post translational modification of CCN3 protein, or inhibition of phosphorylation of CCN3.

Suitably the level or activity of CCN3 or a derivative thereof can be modulated by altering the expression of a nucleic acid encoding CCN3 polypeptide or a derivative thereof in a cell. In a particular embodiment, a nucleic acid, for example a suitable RNAI or antisense nucleic acid, capable of inhibiting transcription of CCN3, can be provided. Suitably the level or activity of CCN3 polypeptide or a derivative thereof can be increased or decreased.

In another embodiment of the method, in the step of providing an agent to said cell, the agent is a nucleic acid encoding CCN3 or a derivative or fragment thereof, and expression of said nucleic acid provides CCN3 polypeptide, a derivative or fragment thereof. The nucleic acid sequence can be operably linked to a promoter and/or provided in a vector.

In another embodiment, a small molecule which is a non-peptide can be provided wherein said small molecule alters the expression or the activity of CCN3, for example the phosphorylation state of CCN3.

In another embodiment a peptide modulator of CCN3, for example, an antibody to CCN3 generated using known methods, including a polyclonal, monoclonal, chimeric, single chain antibody and/or FAb fragment may be provided to alter expression or the phosphorylation state of CCN3.

In a still further embodiment, antisense nucleic acid oligomers, or double stranded RNA mediating RNA interference can be provided to a cell to disrupt transcription, splicing or another function of nucleic acid encoding CCN3 in said cell.

Said cell can be a mammalian cell, for example, a human cell. In particular embodiments said cell is a chronic myeloid leukaemia CML progenitor cell.

The expression of the growth regulator CCN3 has been determined to be reduced in both primary human CML cells and cell lines indicating that downregulation of CCN3 contributes to the phenotype observed in chronic myeloid leukaemia (CML).

In particular embodiments, the level or activity of CCN3 is modulated in vitro.

Suitably, the level of CCN3 can be modulated in vivo in a subject to ameliorate symptoms of a condition of the subject associated with or mediated by BCR-ABL activity.

Suitably said condition can be selected from cancer, haematologic malignancy acute or chronic myeloid leukaemia (CML), Philadelphia positive acute lymphoid leukaemia, myeloproliferative disorders or non-malignant conditions associated with increased myelold cells (for example inflammation).

According to a second aspect of the invention, there is provided the use of CCN3 or a derivative thereof in medicine. Suitably CCN3 or a derivative thereof may be used in a method of treating a condition associated with or mediated by BCL-ABL activity, said method comprising administering a therapeutically effective amount of CCN3 or a derivative thereof to a patient in need thereof.

Suitably conditions which can be treated by administering a therapeutically effective amount of CCN3 or a derivative of CCN3 to a patient in need thereof include cancer, haematologic malignancy, acute or chronic myeloid leukaemia (CML), Philadelphia positive acute lymphoid leukaemia, myeloproliferative disorders or non-malignant conditions associated with increased myeloid cells (for example inflammation).

According to a third aspect of the invention there is provided the use of CCN3 or a derivative thereof in the preparation of a medicament for the treatment of at least one of cancer, acute or chronic myeloid leukaemia (CML), Philadelphia positive acute lymphoid leukaemia, myeloprollferative disorders or non-malignant conditions associated with increased myeloid cells (for example inflammation).

According to a fourth aspect of the present invention there is provided a pharmaceutical composition comprising a nucleic acid which encodes CCN3 or a derivative thereof. In a particular embodiment said composition comprises CCN3 or a derivative thereof, for example a fragment, thereof and a pharmaceutically acceptable carrier or excipient.

Previous studies have found that Notch ligand binds to its receptor and initiates signalling by releasing the intracellular domain of the notch receptor through a cascade of proteolytic cleavages involving alpha (α) and gamma (γ) secretase. The finding that targeting of the γ secretase complex can inactivate Notch signalling has lead to the production of γ secretase inhibitors, for example GSI and LY-411,575. The Inventors have determined that CCN3 interacts with Notch in haematopoietic CML cells. The inventors have determined that γ secretase inhibitors antagonise CCN3 growth regulation in vitro which suggests that γ secretase inhibitors could lead to myeloproliferation. As myeloproliferation could be modulated by the provision of CCN3, it may be advantageous to provide compositions comprising both a γ secretase inhibitor and CCN3. Thus, in particular embodiments of the composition of the invention, the composition further comprises at least one γ secretase inhibitor.

In particular embodiments the composition further comprises inhibitors of Notch ligand binding to the Notch receptor.

The inventors determination that expression of CCN3 correlates with BCR-ABL protein tyrosine kinase activity at an early stage in transformation also provides a means by which treatment of CML using existing medicaments can be measured.

Accordingly a fifth aspect of the present invention provides a method of determining the level of BCR-ABL activity in a subject comprising the steps;
(a) providing a biological sample from a subject,
(b) determining the level of CCN3 or a derivative thereof in said biological sample, and
(c) determining the level of BCR-ABL activity in the subject by correlation of said level of CCN3 or a derivative thereof in the subject with the activity of BCR-ABL.

Accordingly, a sixth aspect of the present invention provides a method of monitoring BCR-ABL kinase activity from a first timepoint to a later timepoint, said method comprising the steps:
providing a first biological sample obtained at the first timepoint, determining the level of CCN3 or a derivative thereof in said biological sample,
providing a second biological sample obtained at the later timepoint,
determining the level of CCN3 or a derivative thereof in said second biological sample, and
determining the difference in the level of CCN3 or a derivative thereof between the first and second biological samples,
wherein a lower concentration at the second timepoint is indicative of increased BCR-ABL kinase activity.

Suitably, the step(s) of determining the level of CCN3 or a derivative thereof can differentiate between phosphorylated and non-phosphorylated CCN3.

Suitably, the step of determining the level of CCN3 can determine the level of CCN3 nucleic acid or CCN3 polypeptide. Suitably the level of nucleic acid can be determined, for example, by probing the sample with a labelled nucleotide sequence capable of hybridising to nucleic acid encoding CCN3, to allow the level of CCN3 nucleic acid to be quantified.

Suitably the level of CCN3 polypeptide can be determined, for example, using antibodies with binding specificity to CCN3 polypeptide and an ELISA assay as would be known to those of skill in the art.

Suitably the methods of the fifth or sixth aspects of the invention can further comprise the step of providing an inhibitor, which includes a candidate inhibitor compound of BCR-ABL expression or activity, wherein an increase in expression or activity of CCN3 or a derivative thereof is indicative that the candidate compound is an inhibitor of BCR-ABL expression or activity.

In particular embodiments the monitoring of the level of CCN3 may be used to monitor the clinical response of a patient to a drug, for example imatinib or the like.

The level of CCN3 can be measured and/or quantified by any suitable means in the art, for example, but not limited to, real time PCR, by immunoassay, for example via an antibody to determine the presence of full or part of the peptide CCN3, for example a monoclonal antibody, the presence of which may be established by exposure to a second labelled monoclonal antibody in a typical ELISA-style assay, via bioassay, or by determining the presence of full or part of a nucleic acid encoding CCN3 using a labelled nucleic acid probe.

Naturally occurring CCN3 is composed of five distinct structural modules; 1) a secretory signal peptide (SP), 2) Insulin like growth factor binding domain (IGFBP), 3) Von Willebrand type C domain (VWC), 4) Thrombospondin type I repeat (TSP I) and 5) Cysteine rich carboxyl terminal (CT).

In studying the relationship between the structure and the function of the growth regulatory protein CCN3 using constructs coding for full-length CCN3 (domains 1-5), domains 2-5 (NH25), domains 3-5 (NH35) and domains 4-5 (NH45), the inventors have surprisingly determined that expression of partial length constructs of CCN3 encoding domains 3 to 5 and domains 2 to 5 showed a significant reduction in tumour colony formation and also in cell proliferation.

Accordingly a seventh aspect of the present invention provides a nucleic acid consisting essentially of:
(a) a nucleic acid which shares at least 80%, at least 85%, at least 90%, at least 92%, at least 95%, at least 97%, at least 98%, at least 99%, at least 99.5%, at least 99.9% or 100% sequence identity to SEQ ID NO 1 wherein said nucleic acid sequence encodes a fragment of CCN3 polypeptide of SEQ ID NO 4;
(b) a nucleic acid sequence which hybridises to the nucleic acid sequence of (a);
(c) a nucleic acid sequences which, but for the degeneracy of the genetic code, would hybridise to the nucleic acid sequence (a).

In particular embodiments the nucleic acid sequence comprises:
(a) a nucleic acid which shares at least 80%, at least 85%, at least 90%, at least 92%, at least 95%, at least 97%, at least 98%, at least 99%, at least 99.5%, at least 99.9% or 100% sequence identity to SEQ ID NO 1 wherein said nucleic acid sequence encodes a fragment of CCN3 polypeptide of SEQ ID NO 4 which does not include a secretory signal peptide (domain 1) of full length CCN3;
(b) a nucleic acid sequence which hybridises to the nucleic acid sequence of (a);
(c) a nucleic acid sequence which, but for the degeneracy of the genetic code, would hybridise to the nucleic acid sequence (a); or
(d) fragments of (a), (b) or (c) wherein said fragments have one or more biological property of CCN3 polypeptide.

In other embodiments the nucleic acid consists essentially of
(a) a nucleic acid sequence or the complementary nucleic acid sequence thereto wherein said nucleic acid sequence shares at least 80%, at least 85%, at least 90%, at least 92%, at least 95%, at least 97%, at least 98%, at least 99%, at least 99.5%, at least 99.9% or 100% sequence identity to SEQ ID NO 3;
(b) a nucleic acid sequence which hybridises to the nucleic acid sequence (a);
(c) a nucleic acid sequence which, but for the degeneracy of the genetic code, would hybridise to the nucleic acid sequence (a); or
(d) fragments of (a), (b) or (c) wherein said fragments encode a polypeptide product which has one or more biological property of domains 3 to 5 of the CCN3 polypeptide.

In other embodiments the nucleic acid consists essentially of
(a) a nucleic acid sequence or the complementary nucleic acid sequence thereto wherein said nucleic acid sequence shares at least 80%, at least 85%, at least 90%, at least 92%, at least 95%, at least 97%, at least 98%, at least 99%, at least 99.5%, at least 99.9% or 100% sequence identity to SEQ ID NO 2;
(b) a nucleic acid sequence which hybridises to the nucleic acid sequence (a);
(c) a nucleic acid sequence which, but for the degeneracy of the genetic code, would hybridise to the nucleic acid sequence (a); or
(d) fragments of (a), (b) or (c) wherein said fragments encode a polypeptide product which has one or more biological property of domains 2 to 5 of the CCN3 polypeptide.

In specific embodiments the nucleic acid consists essentially of
(a) a nucleic acid sequence or the complementary nucleic acid sequence thereto wherein said nucleic acid sequence has a nucleotide sequence SEQ ID NO 3;
(b) a nucleic acid sequence which hybridises to the nucleic acid sequence SEQ ID NO 3;
(c) a nucleic acid sequence which, but for the degeneracy of the genetic code, would hybridise to the nucleic acid sequence SEQ ID NO 3; or
(d) fragments of (a), (b) or (c) wherein said fragments encode a polypeptide product which has one or more biological property of domains 3 to 5 of the CCN3 polypeptide.

In other specific embodiments the nucleic acid consists essentially of
(a) a nucleic acid sequence or the complementary nucleic acid sequence thereto wherein said nucleic acid sequence has a nucleotide sequence SEQ ID NO 2;
(b) a nucleic acid sequence which hybridises to the nucleic acid sequence SEQ ID NO 2;
(c) a nucleic acid sequence which, but for the degeneracy of the genetic code, would hybridise to the nucleic acid sequence SEQ ID NO 2; or
(d) fragments of (a), (b) or (c) wherein said fragments encode a polypeptide product which has one or more biological property of domains 2 to 5 of the CCN3 polypeptide.

Biological properties of domains 3 to 5 of CCN3 include regulation of cell cycle progression, cell proliferation or colony formation. The biological properties can be assayed by various methods known in the art.

As would be appreciated by those of skill in the art, the biological properties of CCN3 or a derivative thereof, for example a fragment, can be suitably assayed using a several established techniques including flow cytometry, clonogenic assays, adhesion assays, investigations of calcium mediated and other cell signalling pathways.

Suitably the nucleic acid can be an orthologue of human CCN3 (SEQ ID NO 1). Preferably said orthologue has at least 80%, more preferably at least 85%, still more preferably at least 90% and most preferably at least 95% sequence Identity with human CCN3. Methods for identifying orthologues are known in the art.

In particular embodiments said nucleic acid sequence can have one or more biological properties of domains 2 to 5 of the protein CCN3.

Suitably the nucleic acid can be SEQ ID NO 2.

Suitably the nucleic acid can be SEQ ID NO 3.

Accordingly an eighth aspect of the present invention provides a polypeptide wherein said polypeptide sequence consists of a fragment of CCN3 of SEQ ID NO 4 wherein said fragment shares at least 80%, at least 85%, at least 90%, at least 92%, at least 95%, at least 97%, at least 98%, at least 99%, at least 99.5%, at least 99.9% or 100% sequence Identity to SEQ ID NO 4.

A fragment of CCN3 is a portion of the CCN3 sequence provided by SEQ ID NO 4. A fragment is not the full length of CCN3 sequence as provided by SEQ ID NO 4.

In particular embodiments said fragment does not include a secretory signal peptide (domain 1) of full length CCN3.

In particular embodiments the polypeptide sequence consists essentially of an amino acid sequence sharing at least 80%, at least 85%, at least 90%, at least 92%, at least 95%, at least 97%, at least 98%, at least 99%, at least 99.5%, at least 99.9% or 100% sequence identity to the amino acid sequence SEQ ID NO 5 or a variant, analog or a fragment of said sequence wherein said polypeptide has one or more biological property of domains 3 to 5 of the CCN3 polypeptide.

In embodiments said polypeptide sequence consists essentially of an amino acid sequence SEQ ID NO 5 or a variant, analog or a fragment of said sequence wherein said polypeptide has one or more biological property of domains 3 to 5 of the CCN3 polypeptide.

In specific embodiment said polypeptide sequence consists essentially of an amino acid sequence SEQ ID NO 5 wherein said polypeptide has one or more biological property of domains 3 to 5 of the CCN3 polypeptide.

In embodiments of the polypeptide, the polypeptide has one or more biological property of domains 2 to 5 of the CCN3 polypeptide.

In suitable embodiments said polypeptide sequence consists essentially of an amino acid sequence sharing at least at least 80%, at least 85%, at least 90%, at least 92%, at least 95%, at least 97%, at least 98%, at least 99%, at least 99.5%, at least 99.9% or 100% sequence identity to the amino acid sequence SEQ ID NO 6 or a variant, analog or a fragment of said sequence wherein said polypeptide has one or more biological property of domains 2 to 5 of the CCN3 polypeptide.

In particular embodiments said polypeptide sequence consists essentially of an amino acid sequence SEQ ID NO 6 or a variant, analog or a fragment of said sequence wherein said polypeptide has one or more biological property of domains 2 to 5 of the CCN3 polypeptide.

In specific embodiments said polypeptide sequence consists essentially of an amino acid sequence SEQ ID NO 6 wherein said polypeptide has one or more biological property of domains 2 to 5 of the CCN3 polypeptide.

Suitably the fragment can be SEQ ID NO 5.

Suitably the fragment can be SEQ ID NO 6.

The peptides of the invention can be produced by use of the corresponding encoding nucleic acid sequences in a suitable expression system.

According to a ninth aspect of the invention there is provided a construct comprising nucleic acid of the seventh aspect of the invention wherein said nucleic acid is operably linked to a promoter.

Nucleic acid of and for use in the present invention can comprise DNA and/or RNA polynucleotides. It can be produced recombinantly, synthetically, or by any means available to those in the art, including cloning using standard techniques.

Suitably nucleic acid can be inserted in an appropriate vector wherein the nucleic acid is operably linked to a control sequence which is capable of providing expression of the nucleic acid in a host cell. A variety of vectors can be used. For example suitable vectors can include viruses (eg. Vaccinia virus, adenovirus, baculovirus etc), yeast vectors, phage, chromosomes, artificial chromosomes, plasmids or cosmid DNA.

According to a tenth aspect of the invention there is provided a vector comprising a construct of the ninth aspect of the invention comprising nucleic acid operably linked to a promoter.

The vectors can be used to introduce the nucleic acid into a host cell. A wide variety of host cells can be used. The host cells can be prokaryotic or ‘eukaryotic’. They include bacteria, eg E. coli yeast, insect cells and mammalian cells. Mammalian cell lines which can be used include Chinese hamster ovary cells, baby hamster kidney cells, NSO mouse melanoma cells, monkey and human cell lines and derivatives thereof and many others. A host cell strain that modulates the expression of, modifies, and/or specifically processes the gene product can be used. Such processing can involve glycosylation, ubiquination, disulfide bond formation and general post-translational modification. For further details relating to known techniques and protocols for manipulation of nucleic acid, for example, in preparation of nucleic acid constructs, mutagenesis, sequencing, introduction of DNA into cells and gene expression, and analysis of proteins, see, for example, Current Protocols in Molecular Biology, 2nd ed., Ausubel et al. eds., JohnWiley & Sons, 1992 and, Molecular Cloning: a Laboratory Manual: 3^{rd} edition Sambrook et al., Cold Spring Harbor Laboratory Press, 2000.

According to a further aspect of the invention, there is provided a cell comprising a vector of the tenth aspect of the invention.

### CCN3 or a Derivative Thereof

In embodiments of the first, second, third, fourth, fifth or sixth aspects of the invention. CCN3 or a derivative thereof may be encoded by a nucleic acid comprising:
(d) a nucleic acid sequence or the complementary nucleic acid sequence thereto wherein said nucleic acid sequence shares 80%, 85%, 90%, 92%, 95%, 97%, 98%, 99%, 99.5%, 99.9% or 100% sequence identity to SEQ ID NO 1;
(e) a nucleic acid sequence which hybridises to the nucleic acid sequence SEQ ID NO 1;
(f) nucleic acid sequences which, but for the degeneracy of the genetic code, would hybridise to the nucleic acid sequence SEQ ID NO 1; or
(g) fragments of (a), (b) or (c) wherein said fragments encode a polypeptide product which has one or more biological properties of domains 3 to 5 of the CCN3 polypeptide.

SEQ ID NO 1

SEQ ID AY082381.1 No 1 Human CCN3 mRNA coding
sequence

| | |
|---|---|
| 1 | gggaaggcga gcagtgccaa tctacagcga agaaagtctc |
| | gtttggtaaa agcgagaggg |
| 61 | gaaagcctga gcatgcagag tgtgcagagc acgagctttt |
| | gtctccgaaa gcagtgcctt |
| 121 | tgcctgacct tcctgcttct ccatctcctg ggacaggtcg |
| | ctgcgactca gcgctgccct |
| 181 | ccccagtgcc cgggccggtg ccctgcgacg ccgccgacct |
| | gcgcccccgg ggtgcgcgcg |
| 241 | gtgctggacg gctgctcatg ctgtctggtg tgtgcccgcc |
| | agcgtggcga gagctgctca |
| 301 | gatctggagc catgcgacga gagcagtggc ctctactgtg |
| | atcgcagcgc ggaccccagc |
| 361 | aaccagactg gcatctgcac ggcggtagag ggagataact |
| | gtgtgttcga tggggtcatc |
| 421 | taccgcagtg gagagaaatt tcagccaagc tgcaaattcc |
| | agtgcacctg cagagatggg |
| 481 | cagattggct gtgtgccccg ctgtcagctg gatgtgctac |
| | tgcctgagcc taactgccca |
| 541 | gctccaagaa aagttgaggt gcctggagag tgctgtgaaa |
| | agtggatctg tggcccagat |
| 601 | gaggaggatt cactgggagg ccttaccctt gcagcttaca |
| | ggccagaagc caccctagga |
| 661 | gtagaagtct ctgactcaag tgtcaactgc attgaacaga |
| | ccacagagtg gacagcatgc |
| 721 | tccaagagct gtggtatggg gttctccacc cgggtcacca |
| | ataggaaccg tcaatgtgag |
| 781 | atgctgaaac agactcggct ctgcatggtg cggccctgtg |
| | aacaagagcc agagcagcca |
| 841 | acagataaga aaggaaaaaa gtgtctccgc accaagaagt |
| | cactcaaagc catccacctg |
| 901 | cagttcaaga actgcaccag cctgcacacc tacaagccca |
| | ggttctgtgg ggtctgcagt |
| 961 | gatggccgct gctgcactcc ccacaatacc aaaaccatcc |
| | aggcagagtt tcagtgctcc |
| 1021 | ccagggcaaa tagtcaagaa gccagtgatg gtcattggga |
| | cctgcacctg tcacaccaac |
| 1081 | tgtcctaaga acaatgaggc cttcctccag gagctggagc |
| | tgaagactac cagagggaaa |
| 1141 | atgtaaccta tcactcaaga agcacaccta cagagcacct |
| | gtagctgctg cgccacccac |
| 1201 | catcaaagga atataagaaa agtaatgaag aatcacgatt |
| | tcatccttga atcctatgta |
| 1261 | ttttcctaat gtgatcatat gaggaccttt catatctgtc |
| | ttttatttaa caaaaaatgt |
| 1321 | aattaactgt aaacttggaa tcaaggtaag ctcaggatat |
| | ggcttaggaa tgacttactt |
| 1381 | tcctgtggtt ttattacaaa tgcaaatttc tataaattta |
| | agaaaacaag tatataattt |
| 1441 | actttgtaga ctgtttcaca ttgcactcat catattttgt |
| | tgtgcactag tgcaattcca |
| 1501 | agaaaatatc actgtaatga gtcagtgaag tctagaatca |
| | tacttaacat ttcattgtac |
| 1561 | aagtattaca accatatatt gaggttcatt gggaagattc |
| | tctattggct ccctttttgg |
| 1621 | gtaaaccagc tctgaacttc caagctccaa atccaaggaa |
| | acatgcagct cttcaacatg |
| 1681 | acatccagag atgactatta cttttctgtt tagttttaca |
| | ctaggaaacg tgttgtatct |
| 1741 | acagtaatga aatgtttact aagtggactg gtgtcataaa |
| | ctttctccat ttaagacaca |
| 1801 | ttgactcctt tccaatagaa agaaactaaa cagaaaactc |
| | ccaatacaaa gatgactggt |
| 1861 | ccctcatagc cctcagacat ttatatattg gaagctgctg |
| | aggcccccaa gttttttaat |
| 1921 | taagcagaaa cagcatatta gcagggattc tctcatctaa |
| | ctgatgagta aactgaggcc |
| 1981 | caaagcactt gcttacatcc tctgatagct gtttcaaatg |
| | tgcattttgt ggaattttga |
| 2041 | gaaaaataga gcaaaatcaa catgactggt ggtgagagac |
| | cacacatttt atgagagttt |
| 2101 | ggaattattg tagacatgcc caaaacttat ccttgggcca |
| | taattatgaa aactcatgat |
| 2161 | caagatatat gtgtatacat acatgtatct ggtttgtcag |
| | gctacaaggt aggctgcaaa |
| 2221 | attaaatcta gacattcttt taatgccacc acacgtgttc |
| | cgcttctctc ttttaaagta |
| 2281 | tttataaaaa tataaattgt acattttgta aaatattatg |
| | tttgatttct ctacttgtca |
| 2341 | tatcactaaa taaacacgat tttattgctg aaaaaaaaaa |
| | aaaaaaaaa |

In particular embodiments of the first, second, third, fourth, fifth, or sixth aspects of the invention a CCN3 polypeptide or a derivative thereof may be encoded by a nucleic acid consisting of:
(h) a nucleic acid sequence or the complementary nucleic acid sequence thereto wherein said nucleic acid sequence shares 80%, 85%, 90%, 92%, 95%, 97%, 98%, 99%, 99.5%, 99.9% or 100% sequence identity to SEQ ID NO 1;
(i) a nucleic acid sequence which hybridises to the nucleic acid sequence SEQ ID NO 1;
(j) nucleic acid sequences which, but for the degeneracy of the genetic code, would hybridise to the nucleic acid sequence SEQ ID NO 1; or
(k) fragments of (a), (b) or (c) wherein said fragments encode a polypeptide product which has one or more biological properties of domains 2 to 5 of the CCN3 polypeptide.

Suitably, said fragments comprise or consist of SEQ ID NO 2 or SEQ ID NO 3.

SEQ ID No 2

Domain 3-5 coding sequence shown in bold and
underlined

| | |
|---|---|
| 1 | gggaaggcga gcagtgccaa tctacagcga agaaagtctc |
| | gtttggtaaa agcgagaggg |
| 61 | gaaagcctga gcatgcagag tgtgcagagc acgagctttt |
| | gtctccgaaa gcagtgcctt |
| 121 | tgcctgacct tcctgcttct ccatctcctg ggacaggtcg |
| | ctgcgactca gcgctgccct |
| 181 | ccccagtgcc cgggccggtg ccctgcgacg ccgccgacct |
| | gcgcccccgg ggtgcgcgcg |
| 241 | gtgctggacg gctgctcatg ctgtctggtg tgtgcccgcc |
| | agcgtggcga gagctgctca |
| 301 | gatctggagc catgcgacga gagcagtggc ctctactgtg |
| | atcgcagcgc ggaccccage |
| 361 | aaccagactg gcatctgcac ggcggtagag ggagataact |
| | gtgtgttcga tggggtcatc |
| 421 | taccgcagtg gagagaaatt tcagacaaga tgcaaattac |
| | agtgcacatg cagagatggg |
| 481 | cagattggct gtgtgccccg ctgtcagctg gatgtgctac |
| | tgcctgagcc taactgccca |
| 541 | gctccaagaa aagttgaggt gcctggagag tgctgtgaaa |
| | agtggatctg tggcccagat |
| 601 | gaggaggatt cactgggagg ccttaccctt gcagattaca |
| | ggccagaagc caccctagga |
| 661 | gtagaagtct ctgactcaag tgtcaactgc attgaacaga |
| | ccacagagtg gacagcatgc |
| 721 | tccaagagct gtggtatggg gttctccacc cgggtcacca |
| | ataggaaccg tcaatgtgag |
| 781 | atgctgaaac agactcggct ctgcatggtg cggccctgtg |
| | aacaagagcc agagcagcca |
| 841 | acagataaga aaggaaaaaa gtgtctccgc accaagaagt |
| | cactcaaagc catccacctg |
| 901 | cagttcaaga actgcaccag cctgcacacc tacaagccca |
| | ggttctgtgg ggtctgcagt |
| 961 | gatggccgct gctgcactcc ccacaatacc aaaaccatcc |
| | aggcagagtt tcagtgctcc |
| 1021 | ccagggcaaa tagtcaagaa gccagtgatg gtcattggga |
| | cctgcacctg tcacaccaac |
| 1081 | tgtcctaaga acaatgaggc cttcctccag gagctggagc |
| | tgaagactac cagagggaaa |
| 1141 | atgtaaccta tcactcaaga agcacaccta cagagcacct |
| | gtagctgctg cgccacccac |
| 1201 | catcaaagga atataagaaa agtaatgaag aatcacgatt |
| | tcatccttga atcctatgta |
| 1261 | ttttcctaat gtgatcatat gaggaccttt catatctgtc |
| | ttttatttaa caaaaaatgt |
| 1321 | aattaactgt aaacttggaa tcaaggtaag ctcaggatat |
| | ggcttaggaa tgacttactt |
| 1381 | tcctgtggtt ttattacaaa tgcaaatttc tataaattta |
| | agaaaacaag tatataattt |
| 1441 | actttgtaga ctgtttcaca ttgcactcat catattttgt |
| | tgtgcactag tgcaattcca |
| 1501 | agaaaatatc actgtaatga gtcagtgaag tctagaatca |
| | tacttaacat ttcattgtac |
| 1561 | aagtattaca accatatatt gaggttcatt gggaagattc |
| | tctattggct ccctttttgg |
| 1621 | gtaaaccagc tctgaacttc caagctccaa atccaaggaa |
| | acatgcagct cttcaacatg |
| 1681 | acatccagag atgactatta cttttctgtt tagttttaca |
| | ctaggaaacg tgttgtatct |
| 1741 | acagtaatga aatgtttact aagtggactg gtgtcataaa |
| | ctttctccat ttaagacaca |
| 1801 | ttgactcctt tccaatagaa agaaactaaa cagaaaactc |
| | ccaatacaaa gatgactggt |
| 1861 | ccctcatagc cctcagacat ttatatattg gaagctgctg |
| | aggcccccaa gttttttaat |
| 1921 | taagcagaaa cagcatatta gcagggattc tctcatctaa |
| | ctgatgagta aactgaggcc |
| 1981 | caaagcactt gcttacatcc tctgatagct gtttcaaatg |
| | tgcattttgt ggaattttga |
| 2041 | gaaaaataga gcaaaatcaa catgactggt ggtgagagac |
| | cacacatttt atgagagttt |
| 2101 | ggaattattg tagacatgcc caaaacttat ccttgggcca |
| | taattatgaa aactcatgat |
| 2161 | caagatatat gtgtatacat acatgtatct ggtttgtcag |
| | gctacaaggt aggctgcaaa |
| 2221 | attaaatcta gacattcttt taatgccacc acacgtgttc |
| | cgcttctctc ttttaaagta |
| 2281 | tttataaaaa tataaattgt acattttgta aaatattatg |
| | tttgatttct ctacttgtca |
| 2341 | tatcactaaa taaacacgat tttattgctg aaaaaaaaaa |
| | aaaaaaaaa |

SEQ ID No 3

Domain 2-5 coding sequence shown in bold and
underlined-excludes signal peptide

| | |
|---|---|
| 1 | gggaaggcga gcagtgccaa tctacagcga agaaagtctc |
| | gtttggtaaa agcgagaggg |
| 61 | gaaagcctga gcatgcagag tgtgcagagc acgagctttt |
| | gtctccgaaa gcagtgcctt |
| 121 | tgcctgacct tcctgcttct ccatctcctg ggacaggtcg |
| | ctgcgactca gcgctgccct |
| 181 | ccccagtgcc cgggccggtg ccctgagacg ccgccgacct |
| | gcgcccccgg ggtgcgcgcg |
| 241 | gtgctggacg gctgctcatg ctgtctggtg tgtgcccgcc |
| | agcgtggcga gagctgctca |
| 301 | gatctggagc catgcgacga gagcagtggc ctctactgtg |
| | atcgcagcgc ggaccccagc |
| 361 | aaccagactg gcatctgcac ggcggtagag ggagataact |
| | gtgtgttcga tggggtcatc |
| 421 | taccgcagtg gagagaaatt tcagccaagc tgcaaattcc |
| | agtgcacctg cagagatggg |
| 481 | cagattggct gtgtgccccg ctgtcagctg gatgtgctac |
| | tgcctgagcc taactgccca |
| 541 | gctccaagaa aagttgaggt gcctggagag tgctgtgaaa |
| | agtggatctg tggcccagat |
| 601 | gaggaggatt cactgggagg ccttaccctt gcagcttaca |
| | ggccagaagc caccctagga |
| 661 | gtagaagtct ctgactcaag tgtcaactgc attgaacaga |
| | ccacagagtg gacagcatgc |
| 721 | tccaagagct gtggtatggg gttctccacc cgggtcacca |
| | ataggaaccg tcaatgtgag |
| 781 | atgctgaaac agactcggct ctgcatggtg cggccctgtg |
| | aacaagagcc agagcagcca |
| 841 | acagataaga aaggaaaaaa gtgtctccgc accaagaagt |
| | cactcaaagc catccacctg |
| 901 | cagttcaaga actgcaccag cctgcacacc tacaagccca |
| | ggttctgtgg ggtctgcagt |
| 961 | gatggccgct gctgcactcc ccacaatacc aaaaccatcc |
| | aggcagagtt tcagtgctcc |
| 1021 | ccagggcaaa tagtcaagaa gccagtgatg gtcattggga |
| | cctgcacctg tcacaccaac |
| 1081 | tgtcctaaga acaatgaggc cttcctccag gagctggagc |
| | tgaagactac cagagggaaa |
| 1141 | atgtaaccta tcactcaaga agcacaccta cagagcacct |
| | gtagctgctg cgccacccac |
| 1201 | catcaaagga atataagaaa agtaatgaag aatcacgatt |
| | tcatccttga atcctatgta |
| 1261 | ttttcctaat gtgatcatat gaggaccttt catatctgtc |
| | ttttatttaa caaaaaatgt |
| 1321 | aattaactgt aaacttggaa tcaaggtaag ctcaggatat |
| | ggcttaggaa tgacttactt |
| 1381 | tcctgtggtt ttattacaaa tgcaaatttc tataaattta |
| | agaaaacaag tatataattt |
| 1441 | actttgtaga ctgtttcaca ttgcactcat catattttgt |
| | tgtgcactag tgcaattcca |
| 1501 | agaaaatatc actgtaatga gtcagtgaag tctagaatca |
| | tacttaacat ttcattgtac |
| 1561 | aagtattaca accatatatt gaggttcatt gggaagattc |
| | tctattggct ccctttttgg |
| 1621 | gtaaaccagc tctgaacttc caagctccaa atccaaggaa |
| | acatgcagct cttcaacatg |
| 1681 | acatccagag atgactatta cttttctgtt tagttttaca |
| | ctaggaaacg tgttgtatct |
| 1741 | acagtaatga aatgtttact aagtggactg gtgtcataaa |
| | ctttctccat ttaagacaca |
| 1801 | ttgactcctt tccaatagaa agaaactaaa cagaaaactc |
| | ccaatacaaa gatgactggt |
| 1861 | ccctcatagc cctcagacat ttatatattg gaagctgctg |
| | aggcccccaa gttttttaat |
| 1921 | taagcagaaa cagcatatta gcagggattc tctcatctaa |
| | ctgatgagta aactgaggcc |
| 1981 | caaagcactt gcttacatcc tctgatagct gtttcaaatg |
| | tgcattttgt ggaattttga |
| 2041 | gaaaaataga gcaaaatcaa catgactggt ggtgagagac |
| | cacacatttt atgagagttt |
| 2101 | ggaattattg tagacatgcc caaaacttat ccttgggcca |
| | taattatgaa aactcatgat |
| 2161 | caagatatat gtgtatacat acatgtatct ggtttgtcag |
| | gctacaaggt aggctgcaaa |
| 2221 | attaaatcta gacattcttt taatgccacc acacgtgttc |
| | cgcttctctc ttttaaagta |
| 2281 | tttataaaaa tataaattgt acattttgta aaatattatg |
| | tttgatttct ctacttgtca |
| 2341 | tatcactaaa taaacacgat tttattgctg aaaaaaaaaa |
| | aaaaaaaaa |

As used herein hybridisation conditions would be readily determined by the skilled artisan based on, for example, the length of the DNA. These conditions can be for example a hybridisation conducted in a solution containing 6*SSC (20*SSC represents 333 mM sodium citrate, 333 mM NaCl), 0.5% SDS and 50% formamide at 42[deg.] C., and then the hybridised products are washed in a solution of 0.1*SSC, 0.5% SDS at 68[deg.] C., or to like conditions.

Suitably the nucleic acid sequences used in the Invention comprise nucleic acid sequences that are at least 80% identical to the naturally occurring nucleic acid sequences which encode domains 3 to 5 of CCN3. Also contemplated are embodiments in which the nucleic acid sequences comprise a sequence at least 80%, preferably 85%, more preferably 90%, more preferably 92%, more preferably 95%, more preferably 97%, more preferably 98%, more preferably 99%, yet more preferably 99.5%, yet more preferably 99.9% and most preferably 100% identical to the originally occurring or native sequences which encode domains 2 to 5 and/or 3 to 5 of CCN3. The percent identity may be determined by visual inspection and/or mathematical calculation or by comparing sequence information using known computer programs such as the GAP computer program. Percentage sequence identity is the percentage of nucleotides or amino acids in a comparison sequence which are identical to the nucleotides or amino acids in the subject sequence, or portion thereof after suitable alignment of the comparison and subject sequences. Various methods of alignment would be known to those of skill in the art.

In embodiments of the first, second, third, fourth, fifth or sixth aspects of the invention, CCN3 or a derivative thereof may comprise of at least 80%, 85%, 90%, 92%, 95%, 97%, 98%, 99%, 99.5%, 99.9% or 100% of the amino acid sequence. SEQ ID NO 4, or a variant, or an analog or a fragment of said sequence wherein said fragment, variant or analog has one or more biological property of domains 2 to 5 of the CCN3 polypeptide.

In embodiments of the first, second, third, fourth, fifth or sixth aspects of the invention, CCN3 or a derivative thereof may consist of at least 80%, 85%, 90%, 92%, 95%, 97%, 98%, 99%, 99.5%, 99.9% or 100% of the amino acid sequence SEQ ID NO 4 or a variant, or analog or a fragment of said sequence wherein said fragment has one or more biological properties of domains 3 to 5 of the CCN3 polypeptide.

SEQ ID NO 4

AAL92490.1 Full-length Human CCN3 protein sequence

| | |
|---|---|
| 1 | mqsvqstsfc lrkqclcltf lllhllgqva atqrcppqcp |
| | grcpatpptc apgvravldg |
| 61 | cscclvcarq rgescsdlep cdessglycd rsadpsnqtg |
| | ictavegdnc vfdgviyrsg |
| 121 | ekfqpsckfq ctcrdgqigc vprcqldvll pepncpaprk |
| | vevpgeccek wicgpdeeds |
| 181 | lggltlaayr peatlgvevs dssvncieqt tewtacsksc |
| | gmgfstrvtn rnrqcemlkq |
| 241 | trlcmvrpce qepeqptdkk gkkclrtkks lkaihlqfkn |
| | ctslhtykpr fcgvcsdgrc |
| 301 | ctphntktiq aefqcspgqi vkkpvmvigt ctchtncpkn |
| | neaflqelel kttrgkm |

In embodiments of the first, second, third, fourth, fifth or sixth aspects of the invention, CCN3 or a derivative thereof may comprise of at least 80%, 85%, 90%, 92%, 95%, 97%, 98%, 99%, 99.5%, 99.9% or 100% of the amino acid sequence SEQ ID NO 5, or a variant, or an analog or a fragment of said sequence wherein said fragment, variant or analog has one or more biological property of domains 3 to 5 of the CCN3 polypeptide.

In embodiments of the first, second, third, fourth, fifth or sixth aspects of the invention, CCN3 or a derivative thereof may consist of at least 80%, 85%, 90%, 92%, 95%, 97%, 98%, 99%, 99.5%, 99.9% or 100% of the amino acid sequence SEQ ID NO 6 or a variant, or analog or a fragment of said sequence wherein said fragment has one or more biological properties of domains 2 to 5 of the CCN3 polypeptide.

Suitably said fragments may comprise or consist of SEQ ID NO 5 or 6.

SEQ ID NO 5

SEQ ID No 5 Domain 3-5 protein sequence residue
104 onwards (marked in bold and underlined)

| | |
|---|---|
| 1 | mqsvqstsfc lrkqclcltf lllhllgq va atgrcppqcp |
| | grcpatpptc apgvravldg |
| 61 | cscclvcarq rgescsdlep cdessglycd rsadpsnqtg |
| | ictavegdnc vfdgviyrsg |
| 121 | ekfqpsckfg ctcrdgqigc vprcqldvll pepncpaprk |
| | vevpgeccek wicgpdeeds |
| 181 | lggltlaayr peatlgvevs daavncieqt tewtacsksc |
| | gmgfstrvtn rnrqcemlkg |
| 241 | trlcmvrpce qepeqptdkk gkkclrtkks lkaihlqfkn |
| | ctslhtykpr fcgvcsdgrs |
| 301 | ctphntktiq aefqcspgqi vkkpvmvigt ctchtncpkn |
| | neaflqelel kttrgkm |

SEQ ID NO 6

SEQ ID No 6 Domain 2-5 protein sequence residue
43 onwards (marked in bold and underlined-
excludes signal peptide)

| | |
|---|---|
| 1 | mqsvqstsfc lrkqclcltf lllhllgqva atqrcppqcp |
| | grcpatpptc apgvravldg |
| 61 | cscclvcarq rgescsdlep cdessglycd rsadpsnqtg |
| | ictavegdnc vfdgviyrsg |
| 121 | ekfqpackfq ctcrdgqigc vprcqldvll pepncpaprk |
| | vevpgeccek wicgpdeeds |
| 181 | lggltlaayr peatlgvevs dssvncieqt tewtacsksc |
| | gmgfstrvtn rnrqcemlkq |
| 241 | trlcmvrpce qepeqptdkk gkkclrtkks lkaihlqfkn |
| | ctslhtykpr fcgvcsdgrc |
| 301 | ctphntktiq aefqcspgqi vkkpvmvigt ctchtncpkn |
| | neaflqelel kttrgkm |

The expression “variant” encompasses peptide sequences which include substitution of amino acids, especially a substitution(s) which is/are known for having a high probability of not leading to any significant modification of the biological activity or configuration, or folding, of the protein. These substitutions are known in the art. For example the group of arginine, lysine and histidine are known interchangeable basic amino acids.

Analogs of the peptides used in the present invention include peptides linked to a coupling partner, e.g. an effector molecule, a label, a drug, a toxin and/or a carrier or transport molecule. Techniques for coupling polypeptides to both peptidyl and non-peptidyl coupling partners are well known in the art.

Analogs further include fusion peptides. Peptides used in the invention may be fused with the constant domain of immunoglobulins (IgA, IgE, IgG, IgM), or portions thereof (CH1, CH2, CH3, or any combination thereof), resulting in chimeric polypeptides. These fusion polypeptides or proteins can facilitate purification and show an increased half-life in vivo. Such fusion proteins may be more efficient in binding and neutralizing other molecules than monomeric polypeptides or fragments thereof alone. Fusion proteins may also include peptides used in the invention fused with albumin, for example recombinant human serum albumin or fragments or variants thereof.

The use of polynucleotides encoding such fusion proteins described herein are also encompassed by the invention.

Analogs for use in the present invention further include reverse- or retro-analogues of natural CCN3 proteins, portions thereof or their synthetic derivatives.

Analogs may also be in the form of multimers. Such multimers may be formed from a multimeric peptide that includes the monomeric unit, and a cleavable site (i.e., an enzymatically cleavable site), and then cleaving the multimeric peptide to yield a desired monomer.

Fragments of CCN3 polypeptide having only part of the amino acids of SEQ ID NO 4, may comprises at least 6, up to 15, preferably at least 25 and more preferably at least 50 contiguous amino acids from SEQ ID NO. 4. A fragment of SEQ ID NO 4 which has one or more biological properties of domains 3 to 5 of the protein CCN3 (SEQ ID NO 5) or domains 2 to 5 of the protein CCN3 (SEQ ID NO 6) can be determined using, for example, by C-terminal serial deletion of CCN3 cDNA.

### Prodrugs

The nucleic acid and polypeptides described herein are intended, at least in some embodiments, to be administered to a human or other mammal to treat or prevent a disorder mediated by BCR-ABL kinase activity. Peptides are typically administered parenterally, and may be readily metabolized by plasma proteases. Oral administration, which is perhaps the most attractive route of administration, may be even more problematic. In the stomach, acid degrades and enzymes break down the peptides. Those peptides that survive to enter the intestine intact are subjected to additional proteolysis as they are continuously barraged by a variety of enzymes, including gastric and pancreatic enzymes, exo- and endopeptidases, and brush border peptidases. As a result, passage of peptides from the lumen of the intestine into the bloodstream can be severely limited.

It will be appreciated that in particular embodiments, the present invention can utilse prodrugs which have been developed to enable parenteral and oral administration of therapeutic peptides or polypeptides.

Peptides or polypeptides can be conjugated to various moieties, such as polymeric moieties, to modify the physiochemical properties of the peptide drugs, for example, to increase resistance to acidic and enzymatic degradation and to enhance penetration of such drugs across mucosal membranes. Polypeptides or peptides can be conjugated with polymeric materials, such as dextrans, polyvinyl pyrrolidones, glycopeptides, polyethylene glycol and polyamino acids. The resulting conjugated polypeptides retain their biological activities and solubility in water for parenteral applications. Polypeptides or peptides can be coupled to polyethylene glycol or polypropropylene glycol having a molecular weight of 500 to 20,000 Daltons to provide a physiologically active non-immunogenic water soluble polypeptide composition. The polyethylene glycol or polypropylene glycol protects the polypeptide from loss of activity and the composition can be injected into the mammalian circulatory system with substantially no immunogenic response. In particular embodiments, a prodrug can be coupled to an oligomer with lipophilic or hydrophilic moieties.

Polypeptides or peptides can also be coupled to polymers, such as polydispersed PEG, for example by, a degradable linkage; using 1, 6 or 1,4 benzyl elimination (BE) strategies; the use of trimethyl lock lactonization (TML); a hydroxy-terminated carboxylic acid linker; or an aryl carbamate. Furthermore free amino, amido, hydroxy and/or carboxylic groups of polypeptides or peptides provide functional groups which can be used to convert the peptides into prodrugs

Enzyme inhibitors may be used to slow the rate of degradation of peptides and polypeptides in the gastrointestinal tract. Alternatively pH may be manipulated to inactivate local digestive enzymes; permeation enhancers may be used to improve the absorption of peptides by increasing their paracellular and transcellular transports; nanoparticles as particulate carriers may be used to facilitate intact absorption by the intestinal epithelium, especially, Peyer's patches, and to increase resistance to enzyme degradation; liquid emulsions may be used to protect the drug from chemical and enzymatic breakdown in the intestinal lumen; and micelle formulations may be used for poorly water-solubulized drugs.

In some cases, the peptides can be provided in a suitable capsule or tablet with an enteric coating, so that the peptide is not released in the stomach. Alternatively, or additionally, the peptide can be provided as a prodrug.

### Pharmaceutical Compositions

The invention further provides pharmaceutical compositions comprising a peptide (or nucleic acid) encoding CCN3 or a derivative or fragment thereof. Pharmaceutical compositions according to the present invention, and for use in accordance with the present invention, may comprise, in addition to active ingredient, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be, for example, oral, intravenous, or topical.

The formulation may be a liquid, for example, a physiologic salt solution containing non-phosphate buffer at pH 6.8-7.6, or a lyophilised powder.

### Dose

The compositions are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is ultimately within the responsibility and at the discretion of doctors, and typically takes account of the disorder to be treated, the condition of the individual patient, the site of delivery, the method of administration and other factors known to practitioners.

### Administration

Peptides of and for use in the present invention may be administered alone but will preferably be administered as a pharmaceutical composition, which will generally comprise a suitable pharmaceutical excipient, diluent or carrier selected dependent on the intended route of administration.

The peptides may be administered to a patient in need of treatment via any suitable route. The precise dose will depend upon a number of factors, including the precise nature of the peptide.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides, copolymers of L-glutamic acid and gamma ethyl-L-glutamate, poly (2-hydroxyethyl-methacrylate) or ethylene vinyl acetate.

Liposomes to deliver polypeptides may be prepared by well-known methods: Ordinarily, liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16th edition, Oslo, A. (ed), 1980.

Targeting therapies may be used to deliver the active agent e.g. peptide more specifically to arterial smooth muscle, by the use of targeting systems such as antibody or cell specific ligands.

### Therapeutic Uses

The peptides and nucleic acids of and for use in the invention may be used in the control and/or treatment of a wide variety of clinical conditions in mammals, including humans. The peptides and methods of the invention may be used in the treatment of any condition or disorder for which reduced cell proliferation and/or colony formation may be useful.

“Treatment” or “therapy” includes any regime that can benefit a human or non-human animal. The treatment may be in respect of an existing condition or may be prophylactic (preventative treatment). Treatment may include curative, alleviation or prophylactic effects.

Preferred features of each aspect of the invention are as for each of the other aspects mutatis mutandis.

The present invention will now be described with reference to the following non-limiting examples and with reference to the figures, wherein:

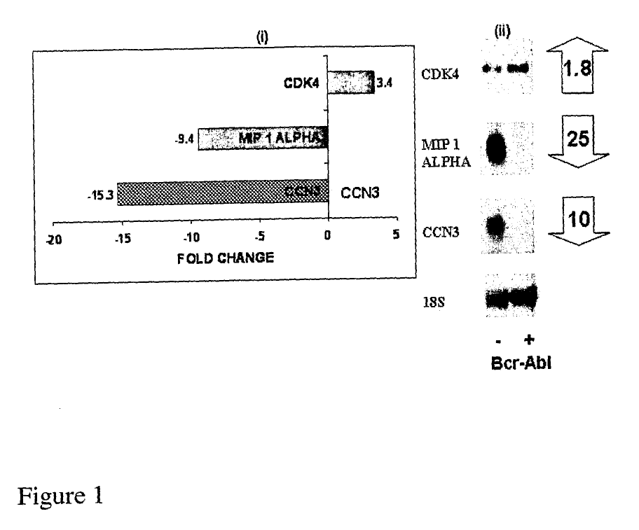

FIG. 1 illustrates differential gene expression as a result of BCR-ABL kinase activity wherein DNA microarray analysis was performed on FDCP-Mix control cells and cells expressing BCR-ABL kinase activity—the microarray identified genes differentially expressed as a result of the BCR-ABL kinase activity, a small subset including CDK4, MIP1 alpha, CCN3 is shown (i). Northern blotting was used to confirm expression of these genes in control cells (−) and BCR-ABL kinase active cells (+) after 24 hours in culture. Fold changes in gene expression are shown (ii);

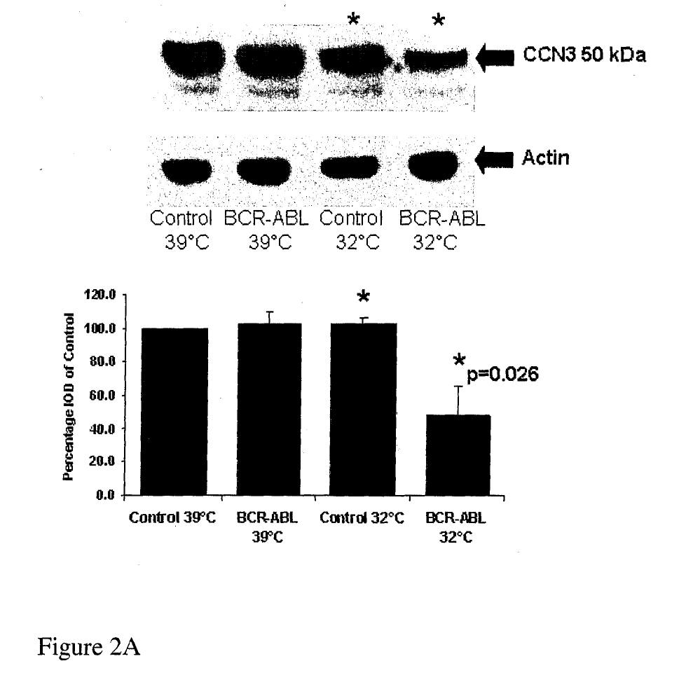

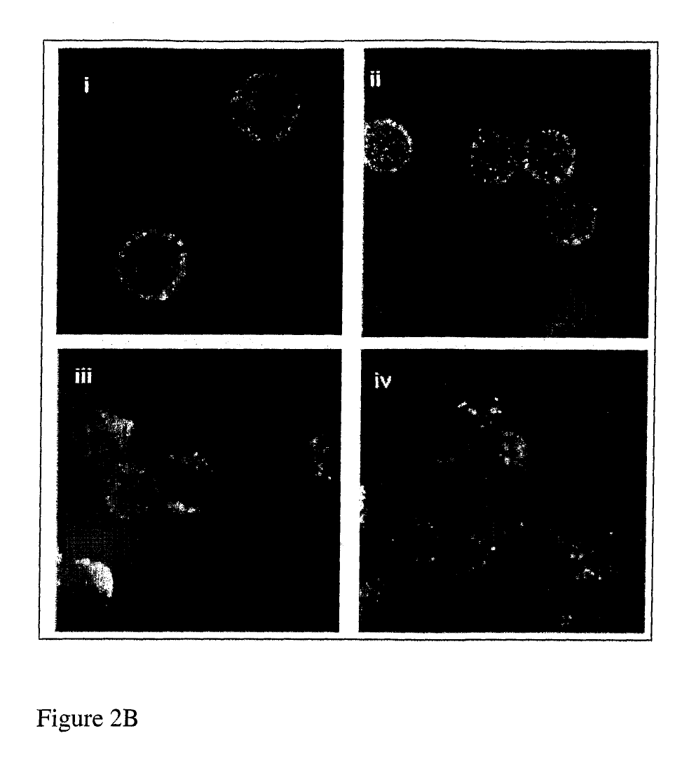

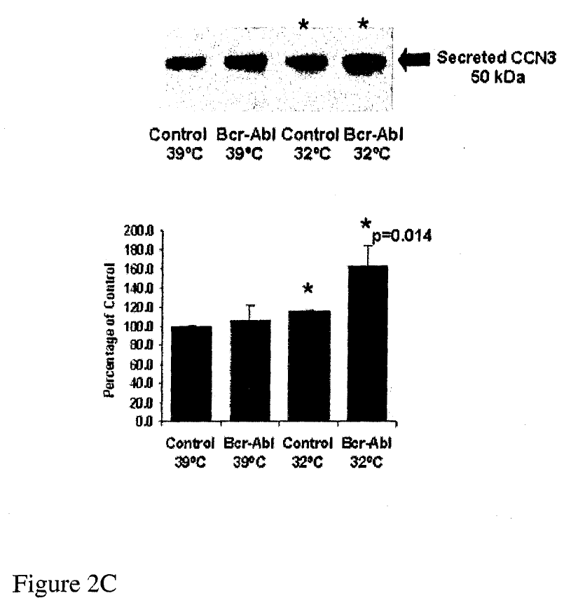

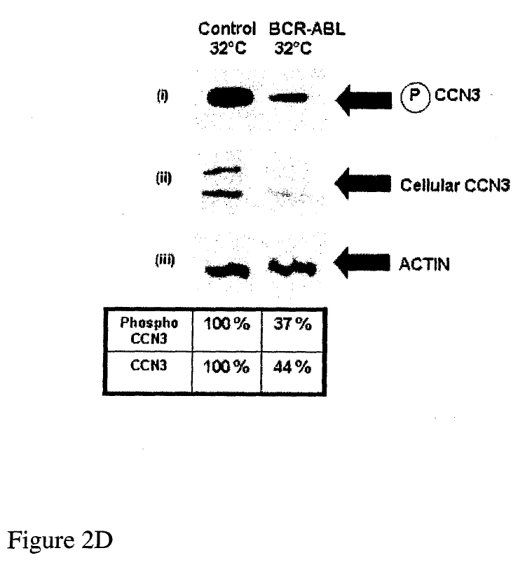

FIG. 2 illustrates CCN3 protein expression is downregulated as a result of BCR-ABL kinase activity wherein A) FDCP-Mix control and ts BCR-ABL cells were grown at the restrictive temperature (39° C.) and permissive temperature for BCR-ABL activity (32° C.) for 24 hours, protein lysates were extracted and probed for CCN3 and actin expression, levels of expression corrected for protein loading are shown as integrated optical densitometry units (IOD); B) Confocal microscopy was also used to identify CCN3 protein (lighter portion) in FDCP-Mix control cells at 39° C. (panel i) and 32° C. (panel ii) and ts BCR-ABL cells at 39° C. (panel iii) and 32° C. (panel iv); propidium iodide nuclear staining (darker portion) is also shown—magnification: ×160; C) Medium in which FDCP-Mix control and ts BCR-ABL cells were grown for 24 hours was collected and probed for secreted CCN3, levels of secreted CCN3 are shown as integrated optical densitometry units (IOD) for comparison; D) Lysates from control cells and BCR-ABL kinase active cells grown at the permissive temperature (32° C.) were immunoprecipitated using CCN3 antibody and probed for (i) phosphotyrosine (ii) CCN3 and (iii) actin expression—Levels of CCN3 and phosphorylated CCN3 detected by Western blot analysis in FDCP-Mix control and BCR-ABL kinase active cells were compared using densitometry as shown;

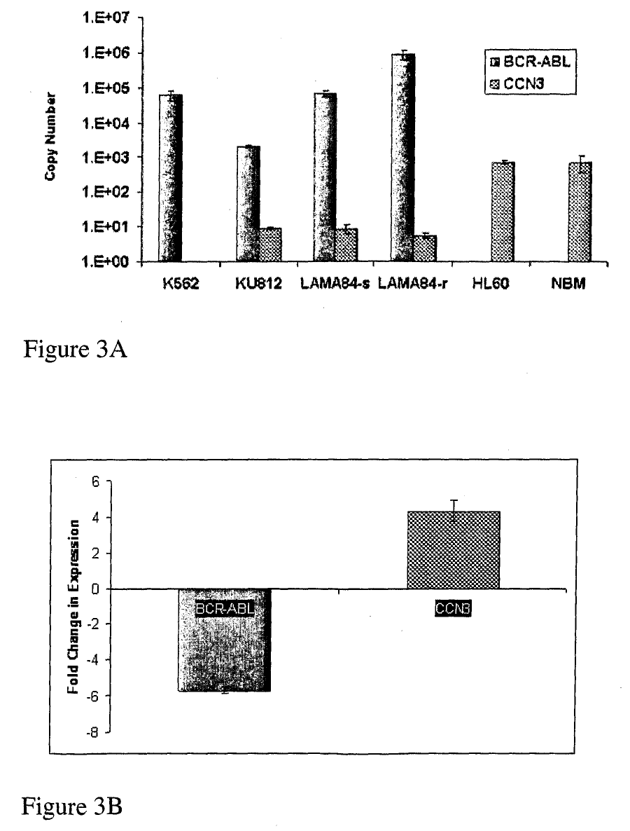

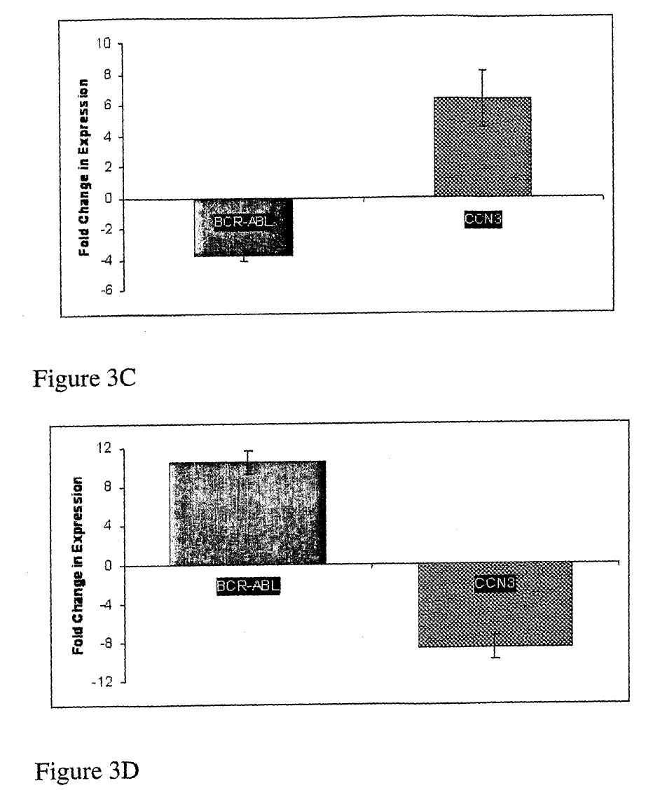

FIG. 3 illustrates CCN3 expression is reduced in human BCR-ABL+ cell lines and can be reversed in K562 cells treated with imatinib or siRNA to BCR-ABL wherein A) RNA was extracted from human CML cell lines (K562, KU812, LAMA), BCR-ABL negative cells (HL60) and Normal Bone Marrow (NBM) and subjected to Real-Time PCR to identify the level of BCR-ABL (solid bars) and CCN3 (hatched bars) expression. Levels of expression are shown as gene copy number; B) K562 cells were treated with imatinib (1 μM) for 96 hours and gene expression was analysed by Real-Time PCR. Fold Changes in BCR-ABL (solid bar) and CCN3 (hatched bar) expression as a result of imatinib treatment are shown; C) K562 cells were treated with siRNA directed to BCR-ABL (0.5 μg/10⁸ cells) for 24 hours—Real-Time PCR was used to identify changes in BCR-ABL (solid bar) and CCN3 (hatched bar) transcripts which are shown as fold change in expression; D) Levels of BCR-ABL (solid bar) and CCN3 (hatched bar) transcripts were compared using Real-time PCR in human CML cells that are sensitive (LAMA84-s) and resistant to imatinib (LAMA84-r)—Alterations In BCR-ABL and CCN3 expression as a result of imatinib resistance are shown as fold changes;

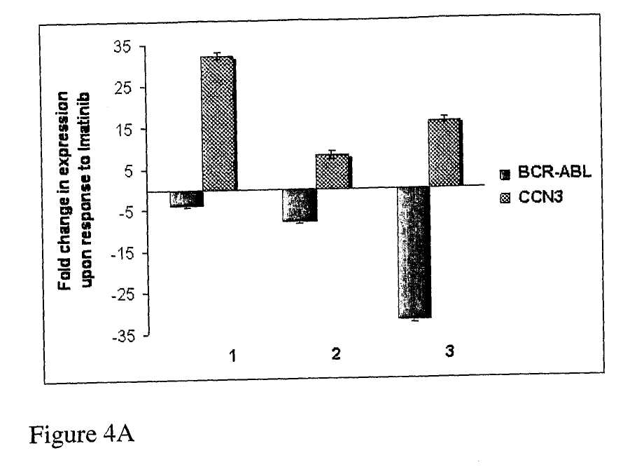

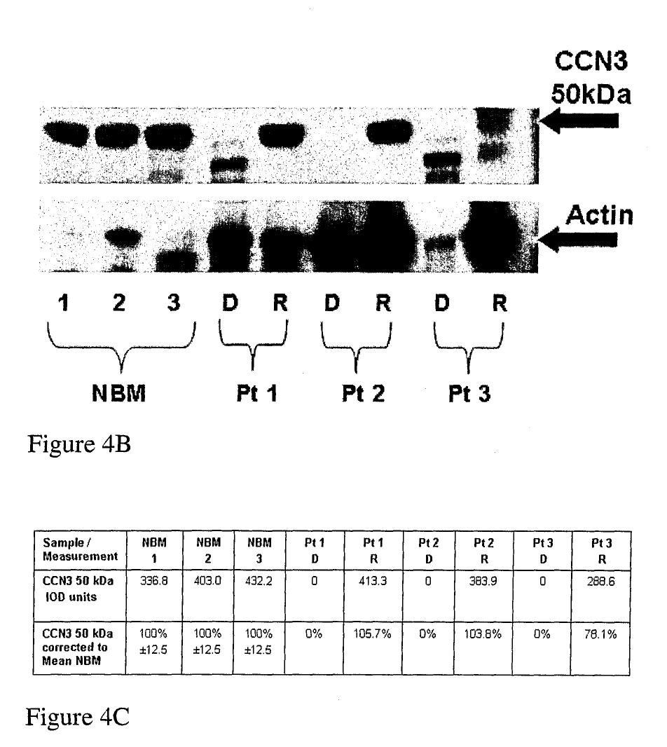

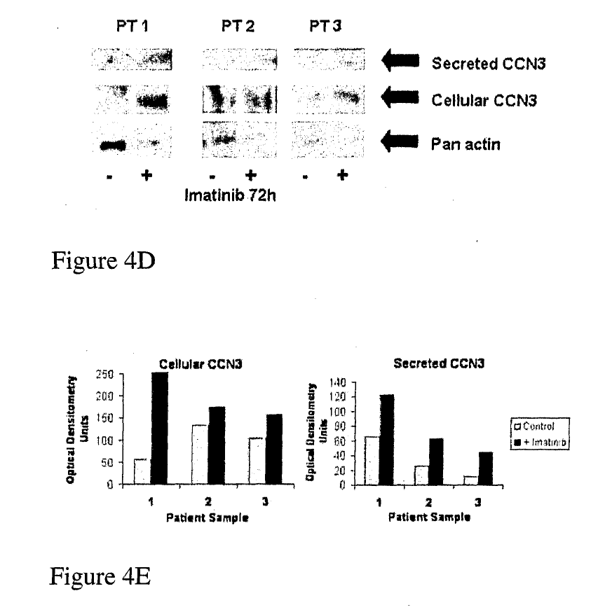

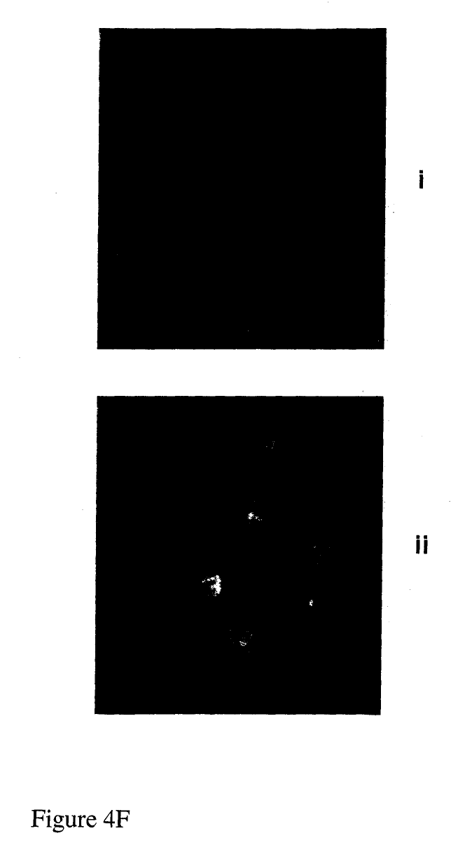

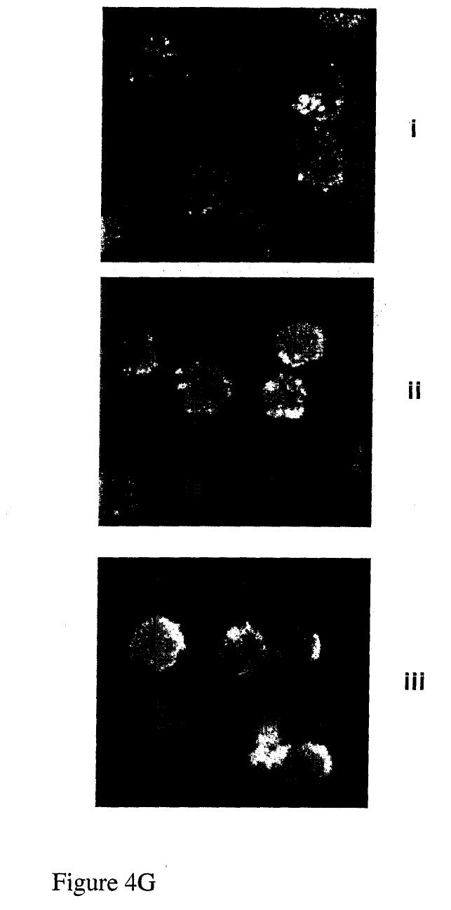

FIG. 4 illustrates CCN3 mRNA and protein is downregulated in primary cells from patients with CML and expression increases upon entering remission wherein A) RNA was extracted from bone marrow samples taken from CML patients at diagnosis and following treatment (patient 1 hematopoietic response, HR, patients 2 & 3 complete cytogenetic response, CCR) and Real-time PCR was performed to determine CCN3 and BCR-ABL mRNA levels. Results are presented as the fold change in BCR-ABL (solid bar) and CCN3 (hatched bar) expression following treatment (mean of three determinations±SD); B) Protein lysates extracted from CML patient bone marrow samples taken at diagnosis (D) and following response to treatment (R) were subjected to Western blot analysis to identify CCN3 protein expression —Three normal bone marrow samples are included for comparison. Patient 1 HR, patient 2 & 3 CCR. C) Optical densitometry was performed on the Western blot from B, the mean signal from NBM was assigned 100% and measurements were expressed as a percentage compared to the signal from NBM; D) CD34+ cells were extracted from the bone marrow of three CML patients at diagnosis and treated for 72 hours in vitro with imatinib (1 μM). Western blot analysis of the medium in which the cells were grown and protein cell lysates was performed to identify CCN3 protein; E) Optical densitometry was performed on the Western blot from D. CCN3 expression in CD34+ cells grown without imatinib were assigned 100%—CCN3 expression in CD34+ cells treated with imatinib for 72 hours was expressed as a percentage compared to cells that were not treated; F) Confocal microscopy was used to detect CCN3 expression in mononuclear cells from a CML patient at diagnosis (panel i) and following a complete cytogenetic response (panel ii)—Magnification: ×80; propidium iodide was used to stain the nuclei; G) Confocal microscopy was used to detect CCN3 protein in CD34+ cells from a CML patient at diagnosis (panel i), normal bone marrow (panel ii) and a patient with thrombocythemia (panel iii). Magnification: ×110; propidium iodide was used to stain the nuclei; and

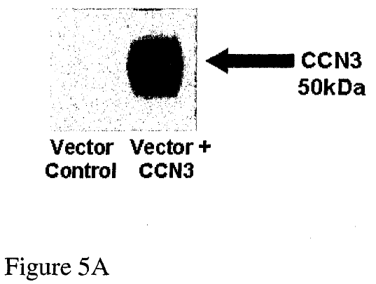

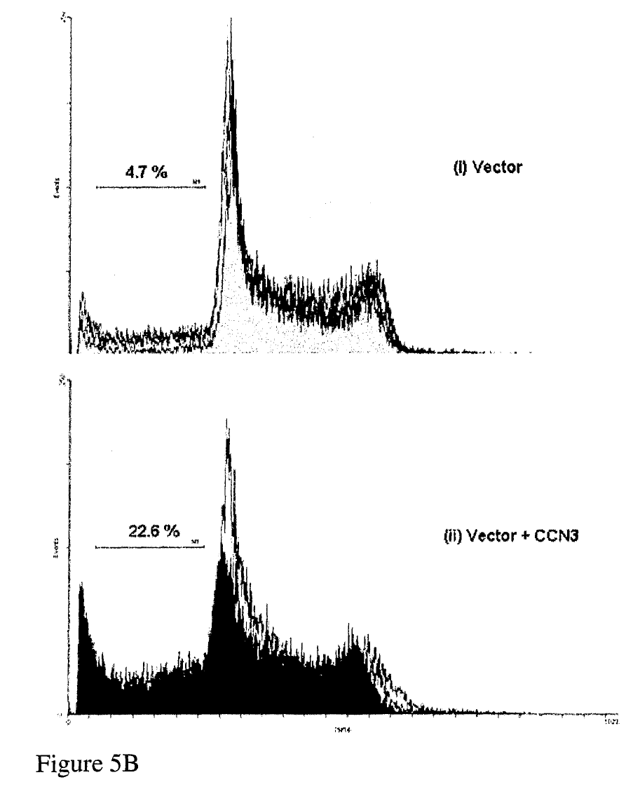

FIG. 5 illustrates increasing CCN3 expression in Bcr-Abl+ cells causes accumulation in the subG₀ area of the cell cycle wherein A) Protein lysates were extracted from K562 cells 24 hours post-transfection with vector alone or vector containing the full-length CCN3 construct—Western blotting was performed to detect CCN3 protein; B) K562 cells transfected with vector alone or vector containing the full-length CCN3 construct were harvested at 24 hours, fixed and stained with propidium iodide—cell cycle profiles were generated and compared for cells containing the vector alone (i) and vector with CCN3 (ii).

### DETAILED DESCRIPTION

### Cell Lines

FDCP-Mix cells transfected with the retroviral vector pM5-neo carrying a p210 ts-BCR-ABL cDNA were used as a model system for CML. The p210 ts-BCR-ABL cDNA encodes a temperature-sensitive mutant of the p210 BCR-ABL which is kinase active when the cells are grown at the permissive temperature of 32° C. Cells transfected with vector only were used as a control. Cells were routinely cultured in Fisher's medium supplemented with pre-selected batches of horse serum (20% v/v), IL-3 (5% v/v) and G418 (50 μg/μL). The cells were subcultured twice weekly to maintain a log phase culture of 2×10⁵ cells/mL and maintained at 39° C. in 5% CO₂ in air. Cells were transferred to the permissive temperature of 32° C. for a minimum of 4 hours prior to experimentation unless otherwise indicated.

HL-60 cells were obtained from the European Collection of Cell Cultures (Salisbury, UK). KU812 and K562 cells were obtained from Deutsche Sammlung von Mikrorganismen und Zellkulturen (DSMZ, GmbH, Braunschweig, Germany). All three of these cell lines were maintained in RPMI-1640 medium supplemented with 10% fetal calf serum (Gibco BRL, Paisley, UK).

LAMA84 imatinib-resistant and sensitive clones (LAMA84-r and LAMA84-s) were provided as a gift from Professor Junia Melo, Imperial College, London. LAMA84-r cells overexpress BCR-ABL and the multidrug resistance P-glycoprotein (Pgp). Imatinib resistant cells were grown in RPMI-1640-10% fetal calf serum supplemented with 1 μM imatinib. Parental sensitive cell lines were maintained in parallel cultures without imatinib.

### Primary CML Samples and Normal Controls

Bone marrow aspirate samples were obtained from normal donors and CML patients at diagnosis or following treatment (as indicated). All human samples were obtained with ethical approval from the local ethics review committee and those involved gave their informed consent for participation in accordance with the Declaration of Helsinki. A complete hematological, response (HR) is defined as white cell count less than 10×10⁹/L with a normal differential count and less than 5% circulating immature cells, platelet count of less than 450×10⁹/L and the disappearance of all signs and symptoms related to CML activity. A major cytogenetic response (MCR) is defined as less than or equal to 35% Philadelphia positive marrow metaphases and a complete cytogenetic response (CCR) as no Philadelphia positive marrow metaphases. Aspirates were collected in RPMI-1640 supplemented with 10% fetal calf serum and containing 100 IU preservative free heparin (Leo Laboratories Ltd, Princes Risborough, UK). Mononuclear cells were separated over Ficoll-Hypaque (Pharmacia Biotech, Uppsala, Sweden) using standard procedures. CD34 positive cells were prepared using the MACS Direct CD34 Progenitor Cell Isolation kit (Miltenyi Biotech Ltd, Bisley, UK).

### DNA Microarray Analysis

All experiments were performed using Affymetrix Mu 6500 oligonucleotide arrays, as described at http://www.affymetrix.com/products/arrays. ts BCR-ABL FDCP-Mlx and control cells were grown at the permissive temperature for 3 hours, 6 hours, 12 hours and 24 hours to capture all events elicited by the oncogene. RNA was isolated by the guanidinium thiocyanate/acid phenol method using TRizol reagent (Gibco BRL). Total RNA from each sample was used to prepare biotinylated target RNA, with minor modifications from the manufacturer's recommendations (http://www.affymetrix.com/support/technical/manual/expression_manual.affx). RNA was purified using RNeasy (Qiagen, Crawley, UK) and mRNA extracted using Oligotex (Qiagen). 10 μg of mRNA from each of the timepoints was pooled and used to generate first-strand cDNA by using a T7-linked oligo(dT) primer. After second-strand synthesis, in vitro transcription was performed with biotinylated UTP and CTP (Enzo Diagnostics, Farmingdale, N.Y.), resulting in approximately 100-fold amplification of RNA. The target cDNA generated from each sample was processed per manufacturer's recommendation using an Affymetrix GeneChip Instrument System (as above). The biotinylated transcripts were then hybridized to an Affymetrix Mu6500 array (Affymetrix®, Santa Clara, Calif.).

Controls were added to 10 μg fragmented cDNA before overnight hybridization. Arrays were then washed and stained with streptavidin-phycoerythrin, before being scanned on an Affymetrix GeneChip scanner. 3′/5′ ratios for GAPDH and beta-actin were confirmed to be within acceptable limits (0.78-0.94), and BloB spike controls were found to be present on all chips, with BioC, BioD and CreX also present in increasing intensity. When scaled to a target intensity of 100 (using Genespring array analysis software), scaling factors for all arrays were within acceptable limits (0.53-1.23), as were background, Q values and mean intensities.

### Northern Blot Analysis

RNA was extracted from ts BCR-ABL FDCP-Mix and control cells after 24 hours in culture using TRIzol reagent (Gibco BRL). Samples were prepared from three separate passages of cells to allow the confirmation of gene expression in triplicate. Northern blotting was performed using a ³²P radlolabeled probe by standard techniques (Thomas PS. Hybridization of denatured RNA and small DNA fragments transferred to nitrocellulose. Proc Natl Acad Sci USA. 1980; 77:5201-5205.). The probe for CCN3 was synthesised by RT-PCR using RNA prepared from the control FDCP-Mix cell line. RNA was reverse transcribed (M-MULV reverse transcriptase, Gibco BRL) and cDNA was amplified using the primers SEQ ID NO 7 5′AAGTCAAGTCTCTGCATCTCTGC3′ and SEQ ID NO 8 5′CTGAGCACCTGTTAAATTTCTCC3′ designed from the sequence X96585 (GenBank). cDNA was denatured for 10 minutes at 94° C. and then amplified over 35 cycles using the following parameters: 1 minute at 94° C., 1 minute at 59° C.; 1 minute at 72° C. A final step of 72° C. for 10 minutes was undertaken to ensure all transcripts were full length. PCR products were visualized after electrophoresis on agarose gels stained with ethidium bromide. Full length product was excised and extracted (QIA Quick Gel Extraction kit, Qiagen) and used for Northern blotting following labeling with radioisotope (Amersham Pharmacia, Chalfont St Giles and NEB, Hitchin, UK).

### Western Blot Analysis

Protein was extracted from cells by suspending in RIPA buffer (1×PBS, 1% Nonidet NP-40, 0.1% SDS) containing a cocktail of protease Inhibitors (Complete Mini Cocktail, Roche Diagnostics Ltd, Lewes, UK) at a concentration of 10⁷ cells/mL and lysing for 10 minutes on ice. Samples were sonicated for 10 seconds to ensure complete lysis, centrifuged at 10,000 g at 4° C. for 10 minutes and the supernatant removed for analysis. Total protein content was determined by the Bradford protein method using the BCA protein assay kit (Pierce, Cramlington, UK). Protein (15 μg) was loaded onto a pre-cast Bis-Tris polyacrylamide gel (10%) using the Novex mini gel system (Invitrogen, Paisley, UK) and subsequently transferred to a PVDF membrane. CCN3 expression was detected using an antibody raised against the C terminus of CCN3. Equivalent protein loading was controlled by monitoring actin expression using a pan-actin antibody (Cell Signalling Technology Inc, MA). Immunoblots were visualised by enhanced chemiluminescence (Supersignal, Pierce). Optical densitometry was performed using the Autochemi™ Sytem (Ultra-Violet Products Ltd., Cambridge, UK) and corrected for protein loading.

CCN3 protein is glycosylated and binds to heparin. Medium in which ts BCR-ABL FDCP-Mix or control cells had been grown for 24 hours was harvested and enriched for CCN3 protein using heparin sepharose. Medium in which CD34+ cells had been grown for 72 hours in the presence or absence of imatinib (1 μM) was harvested and treated in a similar manner. Protein was also extracted from whole cells (2×10⁶) from the same cultures and treated in an identical manner. These preparations are representative of secreted and cellular CCN3 respectively.

The phosphorylation status of CCN3 was examined by immunoprecipitation of CCN3 from whole cell lysates and staining with an antibody to phosphotyrosine. Whole cell lysates (200 μg) were allowed to couple with L59 CCN3 antibody at room temperature for 1 hour. Protein A/G (20 μl, Santa Cruz Biotechnology Inc, Santa Cruz, Calif.) was added, incubated for 1 hour at room temperature and then the protein bound protein A/G complexes were pelleted. The complexes were washed three times in lysis buffer, once in PBS and then resuspended in 50 μL gel loading buffer. Phosphorylated protein was detected using the PY99 antibody (Santa Cruz).

Protein stability was examined by growing ts BCR-ABL FDCP-Mix and control cells in the presence or absence of 1 μg/ml cycloheximide (Sigma). Cells were harvested after 24, 48 or 72 hours and protein extracted as above.

### Confocal Microscopy

Cells were fixed in 50% (v/v) solution of 4% paraformaldehyde in PBS overnight at 4° C. Cells were then pelleted, resuspended in PBS and 2×10⁵ cells were allowed to attach to a silanized slide by evaporation. Slides were transferred to 0.5% (v/v) Triton X-100 (Sigma, Poole, UK) in PBS (PBS-T) for 1 hour and then 5% (v/v) goat serum (Sigma) in PBS-T for 30 minutes to block antibody binding to non-specific sites. Primary antibody (K19M, 1/250) in PBS-T was allowed to bind for 1 hour, unbound antibody was removed by 4×15 minute washes with PBS-T. Slides were immersed in 5% (v/v) goat serum for 30 minutes prior to treatment with secondary antibody, Alexa 488 (1/500, Molecular Probes Inc., Eugene, Oreg.) in PBS-T for 1 hour. Unbound antibody was removed by 4×15 minute washes in PBS-T, treated with 5% (v/v) goat serum in PBS-T for 30 minutes prior to staining with propidium iodide (1 ng/mL, Calbiochem, Nottingham, UK) for 30 minutes. Excess stain was removed by 2×15 minute washes in PBS before being mounted in Vectashield (Vector Laboratories Inc, Burlingame, Calif.) and visualized by a BioRad Microradiance confocal laser scanning microscope (Bio-Rad Laboratories, Hercules, Calif.).

Inhibition of BCR-ABL Gene Expression by si-RNA

K562 cells were nucleofected following the manufacturer's instructions using the Cell Line Nucleofector Kit V, program T-16 (Amaxa GmbH, Cologne, Germany) and transfected with either the appropriate si-RNA, a scrambled si-RNA sequence or a non-silencing fluorescently labeled si-RNA (Amaxa GmbH) to monitor transfection efficiency. A 21-base si-RNA directed against the fusion sequence of BCR-ABL was used to specifically reduce gene expression SEQ ID NO 9 sense 5′GCAGAGUUCAAAAGCCCUUdTdT 3′, SEQ ID NO 10 antisense 5′AAGGGCUUUUGAACUCUGCdTdT 3′ and a scrambled si-RNA sequence was used as control SEQ ID NO 11 sense 5′UUGUACGGCAUCAGCGUUAdTdT 3′, SEQ ID NO 12 antisense 5′UUACGCUGAUGCCGUACAAdTdT 3′. Real-time PCR was performed as previously described to determine the reduction in BCR-ABL mRNA (Scherr M, Battmer K, Winkler T, Heidenreich O, Ganser A, Eder M. Specific inhibition of bcr-abl gene expression by small interfering RNA. Blood. 2003; 101:1566-1569.; Gabert J. Beillard E, van der Velden V H, et al. Standardization and quality control studies of ‘real-time’ quantitative reverse transcriptase polymerase chain reaction of fusion gene transcripts for residual disease detection in leukemia—a Europe Against Cancer program. Leukemia. 2003; 17:2318-2357; Beillard E, Pallisgaard N, van der Velden V H, et al. Evaluation of candidate control genes for diagnosis and residual disease detection in leukemic patients using ‘real-time’ quantitative reverse-transcriptase polymerase chain reaction (RQ-PCR)—a Europe against cancer program. Leukemia. 2003; 17:2474-2486).

### Real-Time Quantitative PCR (RQ-PCR).

RQ-PCR was performed with an ABI PRISM 7700 Sequence Detector which exploits TaqMan probe based chemistry (Applied Biosystems, Foster City, Calif.). The 5′ reporter used was 6-carboxyfluorescein (FAM) and the 3′ quencher was 6-carboxy-tetramethylrhodamine (TAMRA). Primers and probes for CCN3 were designed against GenBank published sequences in association with Primer Express (Applied Biosystems). Primer and probe sets for BCR-ABL were used as recommended by the Europe Against Cancer protocol (Gabert J, Beillard E, van der Velden V H, et al. Standardization and quality control studies of ‘real-time’ quantitative reverse transcriptase polymerase chain reaction of fusion gene transcripts for residual disease detection in leukemia—a Europe Against Cancer program. Leukemia. 2003; 17:2318-2357 and Beillard E, Pallisgaard N, van der Velden V H, et al. Evaluation of candidate control genes for diagnosis and residual disease detection in leukemic patients using ‘real-time’ quantitative reverse-transcriptase polymerase chain reaction (RQ-PCR)—a Europe against cancer program. Leukemia. 2003; 17:2474-2486). The amplification reactions (12.5 μL) contained 59 ng cDNA equivalents (or control), 1× Taqman universal PCR master mix, final concentrations of 5 mM MgCl₂, 0.2 mM deoxyadenosine deoxycytosine deoxyguanosine triphosphate (dATP/dCTP/dGTP), 0.4 deoxyuridine triphosphate (dUTP), 0.125 U AmpliTaq Gold, 2 μM primers (forward and reverse) and 200 nM TaqMan probe. Amplifications were performed following an initial 2-minute incubation at 50° C. to allow uracil-N-glycosylase (UNG) to destroy any contaminating RNA, followed by treatment at 95° C. for 10 minutes to inactivate the UNG enzyme and activate the AmpliTaq Gold DNA polymerase. This was followed by 40 to 45 cycles of denaturing at 95° C. for 15 seconds and annealing/extension at 60° C. for 1 minute. Data were collected and analysed with Sequence Detector v1.6.3 software (Applied Biosystems). Relative quantitative (Q-PCR) data was calculated based on the δδC_{T} method (Ginzinger DG. Gene quantification using real-time quantitative PCR: an emerging technology hits the mainstream. Exp Hematol. 2002; 30:503-512 and Thompson A, Quinn M F, Grimwade D, et al. Global down-regulation of HOX gene expression in PML-RARalpha+acute promyelocytic leukemia identified by small-array real-time PCR. Blood. 2003; 101:1558-1565).

### Expression of CCN3 in K562 Cells

K562 cells were nucleofected following the manufacturer's instructions using the Cell Line Nucleofector Kit V, program T-16 (Amaxa) with 5 μg of either vector (pCb6+, Invitrogen) or vector containing full-length CCN3 sequence. Transfection was performed in triplicate and the functional consequences of CCN3 expression were evaluated 24 hours post-transfection (mean transfected cells 97.9%±0.4). Protein was extracted and Western blotting performed as described above to confirm CCN3 expression. Clonogenicity was determined by plating cells (1×10⁵) in triplicate in methyl cellulose (Stem Cell Technologies Inc, Vancouver, BC) and counted after 7 days on an inverted microscope (magnification: ×40). Cells (5×10⁵) were also fixed in 70% ethanol for flow cytometric analysis of cell cycle status. Fixed cells were resuspended in PBS and incubated in RNase A (100 μg/mL, Qiagen) and propidium iodide (40 μg/mL, Calbiochem) for 30 minutes at 37° C. Samples were then analysed on a Coulter Epics Elite Cell Sorter (Beckman Coulter, High Wycombe, UK). Data was collected on the basis of peak signal versus integral signal to exclude doublets. Analysis was performed on 20,000 events using winMDI software (http://facs.scripps.edu/software.html) and data was generated and then ranked from a common gate in the subG₀ area.

### Effect of Exogenous CCN3 on K562 Cells

K562 cells (1×10⁶ cells/well) were treated with recombinant CCN3 (100 ng/ml, Peprotech EC, London, UK) in vitro for 48 hours. Cells were then fixed in 70% ethanol for flow cytometric analysis of cell cycle status as before.

### Statistical Analysis

Statistical analysis was performed on data from at least 3 experiments using SPSS analytical software 10.0 (http://www.spss.com). Experiments that involved the FDCP-Mix cell line model (kinase active and control cells) were run in parallel and data analysed using the paired samples t-test. Data collected from patient samples were analysed using the non-parametric independent samples method or Mann-Whitney test.

### Example 1

### CCN3 is Downregulated as a Consequence of BCR-ABL Kinase Activity

Microarray analysis, to identify genes whose expression was altered as a consequence of 3-24 hours expression of BCR-ABL PTK activity in multipotent FDCP-Mix cells, was performed. These cells are known to show a suppression of apoptosis over this period but have no autonomous proliferation and no IL-3 production. The DNA microarray analysis identified differential expression of 300 genes as result of BCR-ABL activity and included MIP 1α (downregulated 9.4 fold) and CDK4 (upregulated 3.4 fold) which have previously been associated with leukemogenesis. Expression of CCN3 was downregulated by 15-fold as a consequence of BCR-ABL kinase activity by DNA microarray analysis (FIG. 1 panel i). Northern blot analysis confirmed these observations, demonstrating 25-fold downregulation of MIP 1α, 1.8-fold upregulation of CDK4 and 10-fold downregulation of CCN3 (FIG. 1 panel ii).

### The Expression of CCN3 Protein is Decreased as a Consequence of BCR-ABL Activity

Western blotting of whole cell lysates demonstrated the presence of a doublet corresponding to p50 CCN3, consistent with expression of the full length CCN3 forms (FIG. 2A). CCN3 levels were significantly reduced in the BCR-ABL kinase active cells compared to control cells with a 55% decrease in expression at 24 hours (mean percentage IOD 47.6±18 and 102.8±7.3 respectively, n=3 p=0.026).

Confocal microscopy was employed to examine CCN3 expression (FIG. 2B). CCN3 was clearly detected in the majority of control cells grown at 39° C. and 32° C. (panels i and ii) and in ts BCR-ABL FDCP-Mix cells grown at the restrictive temperature (panels iii). CCN3 expression was significantly reduced in ts BCR-ABL FDCP-Mix cells grown at the permissive temperature (kinase active, panel iv). The majority of cells were negative although a small number of positively staining vesicles could still be observed in occasional cells (mean fluorescent intensity: 2.6±0.65×10⁵, n=5). The decreased CCN3 fluorescence in the kinase active cells was significantly reduced in comparison to the control or kinase inactive cells (mean fluorescence intensity: 4.6±0.87×10⁵, p=0.043). The fluorescence intensity in both the BCR-ABL kinase active or inactive cells was strongest at the cell periphery where it was concentrated into globular or vesicular like structures resembling those for exportation from the cell, a phenomenon known to occur with CCN3.

The medium in which these cells had been grown from kinase active or control FDCP-Mix cells was prepared and subjected to Western blotting (FIG. 2C). This showed that the level of secreted CCN3 was increased in BCR-ABL kinase active cells (BCR-ABL 32° C.) in comparison to control cells grown at 39° C. and 32° C. and in ts BCR-ABL FDCP-Mix cells grown at the restrictive temperature (BCR-ABL 39° C.). The increased secretion of CCN3 in kinase active cells compared to control cells (mean percentage IOD 163.8±20.8 and 116.3±1.0 respectively, n=3 p=0.034) was indicative that regulation of CCN3 expression within cells may be regulated by post-translational processing as well as transcription. Critically, protein stability did not appear to play a part in the decreased cellular protein levels observed as no evidence of protein degradation over a period of 72 hours was found.

The phosphorylation status of intracellular CCN3 in control and BCR-ABL active FDCP-Mix was examined by immunoprecipitating CCN3 and western blotting with antiphosphotyrosine antibodies (FIG. 2D). CCN3 was strongly phosphorylated in control cells and weakly phosphorylated in BCR-ABL kinase active cells (i). The reduction of phosphorylation detected in kinase active cells was considered to be due to the overall decrease in CCN3 expression in BCR-ABL kinase active cells (ii). The cell lysates were tested for actin expression (iii) to ensure equal protein loading.

### CCN3 is Downregulated in Human CML Cell Lines

To confirm the effect of BCR-ABL expression on CCN3 in human cells, real time PCR was employed which showed strong expression of BCR-ABL and weak or absent expression of CCN3 in CML cell lines (FIG. 3A). In K562, KU812 and LAMA84-s cell lines BCR-ABL expression was high whilst CCN3 expression was low. CCN3 expression in the BCR-ABL negative cell line, HL-60, and normal bone marrow (NBM) was high (n=3). Treatment of the CML cell lines with imatinib or si-RNA against BCR-ABL significantly reduced BCR-ABL and increased CCN3 expression. For example K562 cells treated with 1 μM imatinib for 96 hours showed a 5.9 fold decrease in BCR-ABL expression and a 4.2 fold increase in CCN3 expression, as shown in FIG. 3B (mean Ct change 2.5±0.1 and 2.1±0.2 for BCR-ABL and CCN3 respectively, n=3, p=0.001). Treatment of K562 cells with si-RNA directed against BCR-ABL resulted in a 3.7 fold decrease in BCR-ABL and 6.1 fold increase in CCN3, as shown in FIG. 3C (mean Ct change 1.9±0.2 and 2.6±0.5 for BCR-ABL and CCN3 respectively, n=3, p=0.001). The difference in BCR-ABL and CCN3 expression in imatinib resistant cells, LAMA84-r, in comparison to the sensitive LAMA84-s cells is shown (FIG. 3D). Cumulative resistance to imatinib in vitro due to a 10-fold increase in BCR-ABL expression showed a corresponding 9-fold decrease in CCN3 expression (mean Ct change 3.4±0.1 and 3.1±0.3 for BCR-ABL and CCN3 respectively, n=3, p=0.001). Furthermore, in order to analyse whether there was a relationship between BCR-ABL expression and the secretion of CCN3 in a human cell line (K562) siRNA was employed to decrease expression of BCR-ABL and measured the extracellular CCN3 levels. It was determined that there was a significant decrease of 33±5% in secreted levels of CCN3 protein resulting from siRNA expression (p=0.038, n=3). This demonstrates that downregulation of CCN3 is a direct consequence of BCR-ABL activation in human cells as well as murine cells.

### The Expression of CCN3 Protein is Decreased in Primary Human CML Cells

To determine if CCN3 expression was altered in primary CML cells, real time PCR was used to measure CCN3 and BCR-ABL expression in three CML patients at diagnosis and following treatment with imatinib; one patient had a hematopoietic response and two patients had a complete cytogenetic response. In each of the patients a fall in BCR-ABL expression was associated with a reciprocal rise in CCN3 (FIG. 4A). Since CCN3 expression is highest in BCR-ABL negative cells it reflects hematologic response. There does not appear to be a direct correlation between the decrease in BCR-ABL expression and increased CCN3 transcripts possibly reflecting the heterogeneity of the cell types present. CCN3 protein expression was then examined using Western blotting of whole cell lysates of normal bone marrow and bone marrow from three CML patients taken at diagnosis and following response to therapy. Expression of full length CCN3 was detected in all three normal bone marrow samples. A lower molecular weight form of CCN3 (45 kDa) was weakly detectable in two of the patients at diagnosis returning to a normal expression pattern on response to treatment (FIG. 4B). Optical densitometry of the blot from FIG. 4B, showed patients responding to treatment have levels of CCN3 comparable with NBM (FIG. 4E). CD34+ cells taken from three CML patients at diagnosis were treated with imatinib for 72 hours in vitro and a significant increase in both cellular and secreted CCN3 expression was demonstrated in all three samples (FIG. 4D). Optical densitometry of the blot from FIG. 4D shows that treating CD 34+CML cells with imatinib increases CCN3 protein expression (FIG. 4E). CCN3 expression was also examined in primary human cells by confocal microscopy. Mononuclear cells from a CML patient at diagnosis showed only occasional weakly staining cells (FIG. 4F, panel i) but on entering complete cytogenetic remission the majority of cells stained positively for CCN3 (panel ii). Similarly, CD34+ cells from a patient with CML at diagnosis (FIG. 4G panel i) showed a modest amount of staining compared to CD34+ cells from a normal marrow (panel ii). CD34+ cells from a patient with thrombocythemia (panel iii) exhibited a normal expression pattern for CCN3 consistent with the changes in CCN3 being dependent on BCR-ABL activity rather than a reflection of myeloproliferation.

### Increasing CCN3 Expression in BCR-ABL+ Cells

To further investigate the role of CCN3 expression human K562 cells were transfected with a full-length CCN3 construct. Western blot analysis confirmed a strong increase in CCN3 protein in cells transfected with the vector containing the CCN3 construct in comparison to cells transfected with vector alone (FIG. 5A). Flow cytometry was used to perform cell cycle analysis. K562 cells transfected with vector containing the full-length CCN3 construct (FIG. 5B panel ii) showed significantly more cells accumulating in the subG₀ area of the cell cycle than cells that had been transfected with vector alone (FIG. 5B panel i, mean subG₀ 21.8%±0.7 and 9.9%±4.6 respectively p=0.028, n=3). The ability of transfected cells to form colonies on methyl cellulose was also assessed. Colony formation of K562 cells transfected with vector containing the CCN3 construct was significantly reduced by one third in comparison to cells transfected with vector alone (p=0.027, n=3).

The ability of BCR-ABL positive cells to respond to exogenous CCN3 was tested by incubating K562 cells with recombinant CCN3 for 48 hours. Flow cytometry demonstrated a significant increase in the sub-G₀ peak when cells were treated with 100 ng/ml CCN3 (increased from 9.3±3.9 to 23.7±6.9%, p=0.014).

### Example 2

The functional consequence of expressing CCN3 variants in K562 cells was investigated and the relationship between the structure and the function of the CCN3 growth regulatory protein was studied using constructs coding for full-length CCN3 (domains 1-5), domains 2-5 (NH25), domains 3-5 (NH35) and domains 4-5 (NH45). K562 cells were transfected using the Amaxa cell line nucleofector kit with either vector alone (pCb6+) or vector containing the appropriate construct. The functional consequences of expression were evaluated using flow cytometry, colony formation in methyl cellulose and MTT assay.

Expression of full-length CCN3 in K562 cells showed a significant accumulation of cells in the sub G0 phase of cell cycle at 24 h (mean subG0 21.8%±0.7 compared to control 9.9%±4.6, p=0.028). Full-length CCN3 did not alter cell proliferation capacity by MTT assay at 48 h but reduced colony formation by 33% after 7 days. Expression of the partial length constructs NH25 and NH35 showed a significant increase in cells within the subG0 area and reduced colony formation by 20% (p=0.04) and 25% (p=0.036) respectively, when compared to control cells. In contrast to expression of full length CCN3, these constructs also significantly reduced cell proliferation (77.4%±0.36; p=0.001 and 81.6±6.2%; p=0.036 of control). Expression of the NH45 construct did not significantly alter cell cycle profile, cell proliferation or colony formation.

This study demonstrated that CCN3 expression regulates cell proliferation, cell cycle progression and colony formation. The IGFBP and VWC modules of CCN3 are key mediators of negative growth regulation. BCR-ABL expression in CML downregulates CCN3 causing disregulation of these mechanisms and properties consistent with the CML phenotype.

Various modifications may be made to the invention herein described without departing from the scope of the claims.

1. A method of modulating BCR-ABL activity said method comprising modulating the level or activity of CCN3 or a derivative thereof, wherein CCN3 includes CCN3 polypeptide, a nucleic acid encoding CCN3 or a fragment thereof, thereby modulating the level or activity of BCR-ABL.

2. A method of treating a condition associated with or mediated by BCR-ABL activity, said method comprising administering a therapeutically effective amount of CCN3 or a derivative thereof to a patient in need thereof.

3. (canceled)

4. A method as claimed in claim 2 wherein the condition is selected from the group consisting of cancer, acute or Chronic myeloid leukaemia (CML), Philadelphia positive acute lymphoid leukaemia, myeloproliferative disorders or non-malignant conditions associated with increased myeloid cells.

5. A pharmaceutical composition comprising CCN3 or a derivative thereof.

6. A method of determining the level of BCR-ABL activity in a subject comprising the steps;
(a) providing a biological sample from a subject,
(b) determining the level of CCN3 or derivative thereof in said biological sample, and
(c) determining the level of BCR-ABL in the subject by correlation of said level of CCN3 or derivative thereof in the subject with the activity of BCR-ABL.

7. A method of monitoring BCR-ABL kinase activity from a first timepoint to a later timepoint, said method comprising the steps:
providing a first biological sample obtained at the first timepoint,
determining the level of CCN3 or a derivative thereof in said biological sample,
providing a second biological sample obtained at the later timepoint,
determining the level of CCN3 or a derivative thereof in said second biological sample, and
determining the difference in the level of CCN3 or a derivative thereof between the first and second biological samples,
wherein a lower concentration at the second timepoint is indicative of increased BCR-ABL kinase activity.

8. A nucleic acid consisting essentially of:
(a) a nucleic acid which shares at least 80% sequence identity to SEQ ID NO 1 wherein said nucleic acid sequence encodes a fragment of CCN3 polypeptide of SEQ ID NO 4;
(b) a nucleic acid sequence which hybridises to the nucleic acid sequence of (a);
(c) a nucleic acid sequence which, but for the degeneracy of the genetic code, would hybridise to the nucleic acid sequence (a).

9. A nucleic acid sequence as claimed in claim 8 wherein the nucleic acid comprises:
(a) a nucleic acid which shares at least 80% sequence identity to SEQ ID NO 1 wherein said nucleic acid sequence encodes a fragment of CCN3 polypeptide of SEQ ID NO 4 which does not include a secretory signal peptide (domain 1) of full length CCN3;
(b) a nucleic acid sequence which hybridises to the nucleic acid sequence of (a);
(c) a nucleic acid sequence which, but for the degeneracy of the genetic code, would hybridise to the nucleic acid sequence (a); or
(d) fragments of (a), (b) or (c) wherein said fragments have one or more biological property of CCN3 polypeptide.

10. A nucleic acid sequence as claimed in claim 8 consisting essentially of
(a) a nucleic acid sequence or the complementary nucleic acid sequence thereto wherein said nucleic acid sequence shares 80% sequence identity to SEQ ID NO 3;
(b) a nucleic acid sequence which hybridises to the nucleic acid sequence (a);
(c) a nucleic acid sequence which, but for the degeneracy of the genetic code, would hybridise to the nucleic acid sequence (a); or
(d) fragments of (a), (b) or (c) wherein said fragments encode a polypeptide product which has one or more biological property of domains 2 to 5 of the CCN3 polypeptide.

11. A nucleic acid as claimed in claim 8 consisting essentially of
(a) a nucleic acid sequence or the complementary nucleic acid sequence thereto wherein said nucleic acid sequence shares 80% sequence identity to SEQ ID NO 2;
(b) a nucleic acid sequence which hybridises to the nucleic acid sequence (a);
(c) a nucleic acid sequence which, but for the degeneracy of the genetic code, would hybridise to the nucleic acid sequence (a); or
(d) fragments of (a), (b) or (c) wherein said fragments encode a polypeptide product which has one or more biological property of domains 3 to 5 of the CCN3 polypeptide.

12. A nucleic acid sequence as claimed in claim 8 consisting essentially of
(a) a nucleic acid sequence or the complementary nucleic acid sequence thereto wherein said nucleic acid sequence has a nucleotide sequence SEQ ID NO 3;
(b) a nucleic acid sequence which hybridises to the nucleic acid sequence SEQ ID NO 3;
(c) a nucleic acid sequence which, but for the degeneracy of the genetic code, would hybridise to the nucleic acid sequence SEQ ID NO 3; or
(d) fragments of (a), (b) or (c) wherein said fragments encode a polypeptide product which has one or more biological property of domains 2 to 5 of the CCN3 polypeptide.

13. A nucleic acid as claimed in claim 8 consisting essentially of
(a) a nucleic acid sequence or the complementary nucleic acid sequence thereto wherein said nucleic acid sequence has a nucleotide sequence SEQ ID NO 2;
(b) a nucleic acid sequence which hybridises to the nucleic acid sequence SEQ ID NO 2;
(c) a nucleic acid sequence which, but for the degeneracy of the genetic code, would hybridise to the nucleic acid sequence SEQ ID NO 2; or
(d) fragments of (a), (b) or (c) wherein said fragments encode a polypeptide product which has one or more biological property of domains 3 to 5 of the CCN3 polypeptide.

14. A polypeptide wherein said polypeptide sequence consists of a fragment of CCN3 of SEQ ID NO 4 wherein said fragment shares at least 80% sequence identity to SEQ ID NO 4.

15. A polypeptide as claimed by claim 14 wherein said fragment does not include a secretory signal peptide (domain 1) of full length CCN3.

16. A polypeptide as claimed by claim 14 wherein said polypeptide sequence consists essentially of an amino acid sequence sharing at least 80% sequence identity to the amino acid sequence SEQ ID NO 5 or a variant, analog or a fragment of said sequence wherein said polypeptide has one or more biological property of domains 3 to 5 of the CCN3 polypeptide.

17. A polypeptide as claimed by claim 16 wherein said polypeptide sequence consists essentially of an amino acid sequence SEQ ID NO 5 or a variant, analog or a fragment of said sequence wherein said polypeptide has one or more biological property of domains 3 to 5 of the CCN3 polypeptide.

18. A polypeptide as claimed by claim 16 wherein said polypeptide sequence consists essentially of an amino acid sequence SEQ ID NO 5 wherein said polypeptide has one or more biological property of domains 3 to 5 of the CCN3 polypeptide.

19. A polypeptide as claimed in claim 16 having one or more biological property of domains 2 to 5 of the CCN3 polypeptide.

20. A polypeptide as claimed by claim 14 wherein said polypeptide sequence consists essentially of an amino acid sequence sharing at least 80% sequence identity to the amino acid sequence SEQ ID NO 6 or a variant, analog or a fragment of said sequence wherein said polypeptide has one or more biological property of domains 2 to 5 of the CCN3 polypeptide.

21. A polypeptide as claimed by claim 14 wherein said polypeptide sequence consists essentially of an amino acid sequence SEQ ID NO 6 or a variant, analog or a fragment of said sequence wherein said polypeptide has one or more biological property of domains 2 to 5 of the CCN3 polypeptide.

22. A polypeptide as claimed by claim 14 wherein said polypeptide sequence consists essentially of an amino acid sequence SEQ ID NO 6 wherein said polypeptide has one or more biological property of domains 2 to 5 of the CCN3 polypeptide.

23. A vector comprising a nucleic acid as claimed by any one of claims 8 to 13 wherein said nucleic acid is operably linked to a promoter.

24. A cell comprising a vector as claimed by claim 23.
